(19) United States
(12) Patent Application Publication
(10) Pub. No.: US 2011/0257211 A1
(43) Pub. Date: Oct. 20, 2011

(54) Small-Molecule Choline Kinase Inhibitors as Anti-Cancer Therapeutics

(75) Inventors: Pooran Chand, Birmingham, AL (US); Jason A. Chesney, Louisville, KY (US); Brian F. Clem, Louisville, KY (US); Gilles H. Tapolsky, Louisville, KY (US); Sucheta Telang, Louisville, KY (US); John O. Trent, Louisville, KY (US)

(73) Assignees: ADVANCED CANCER THERAPEUTICS, LLC, Louisville, KY (US); UNIVERSITY OF LOUISVILLE RESEARCH FOUNDATION, INC., Louisville, KY (US)

(21) Appl. No.: 12/975,631

(22) Filed: Dec. 22, 2010

Related U.S. Application Data
(60) Provisional application No. 61/324,441, filed on Apr. 15, 2010.

(51) Int. Cl. A61K 31/506; C07D 417/12; C07D 401/12; C07D 401/04; C07D 403/12; A61P 35/00; A61K 31/427; A61K 31/4439; A61K 31/4709; A61K 31/428; A61K 31/454; C12N 9/99; C07D 249/12; A61K 31/4196

(52) U.S. Cl. 514/275; 548/264.4; 548/195; 546/272.4; 546/167; 548/163; 544/331; 546/210; 514/383; 514/371; 514/341; 514/314; 514/367; 514/326; 435/184

(57) ABSTRACT

Small molecule choline kinase inhibitors having the following formula:

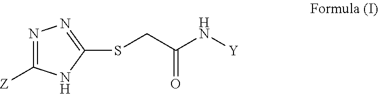

are provided herein. Also provided herein are pharmaceutical compositions containing Formula I compounds, together with methods of treating cancer, methods of inhibiting choline kinase enzymatic activity, and methods of treating tumors by administering an effective amount of a Formula I compound.

### RELATED APPLICATION

This Application claims priority under 35 U.S.C. §119(e) to U.S. Provisional Application Ser. No. 61/324,441, filed Apr. 15, 2010, which application is incorporated by reference herein in its entirety.

### FIELD OF THE INVENTION

The presently-disclosed subject matter relates to small-molecule inhibitors of choline kinase and methods of using the same. In particular, the presently-disclosed subject matter relates to substituted aminocarbonylmethylthio triazoles as choline kinase inhibitors and methods of using these inhibitors to reduce the enzymatic activity of choline kinase and/or treat cancer and tumors.

### BACKGROUND OF THE INVENTION

Choline Kinase (ChoK) is a phosphotransferase which acts by phosphorylating choline to phosphocholine (PCho) as the first enzyme of the phospatidylcholine (PC) synthetic pathway (also known as the Kennedy pathway). Adenosine-triphosphate (ATP) is the phosphate group donor.

Other research has pointed to a role for activated choline kinase as a metabolic requirement for neoplastic growth and survival. Insulin, platelet-derived growth factor, fibroblast growth factor, epidermal growth factor, prolactin, estrogens and hypoxia-inducible factor-1α appear to be needed for the survival, growth and invasiveness of human cancers, and have all been typically found to stimulate choline kinase activity and increase intracellular phosphocholine. Growth factors can engage receptor-tyrosine kinases which stimulate two key signal transducers, the small GTPase Ras and the lipid kinase phosphatidylinositol-3-OH kinase (PI3K). These signal transducers then can stimulate an intersecting network that activates untethered cell growth, survival and invasiveness without influence from environmental cues and, when mutated, initiate tumors in humans.

In addition, oncogenic transformation mediated by Ras oncogenes induces high choline kinase activity levels resulting in an abnormal increase in the intracellular levels of its product, PCho. Ras gene proto-oncogenes encode a protein family of small membrane-bound GTPases which appear to be involved in cellular signal transduction from outside the cell to inside the nucleus. Activation of Ras signaling causes cell growth, division, terminal differentiation and senescence. Mutations in Ras are heavily implicated in the development of cancers. It is hypothesized that mutations may permanently activate Ras. Ras oncogenic transforming potential is acquired with point amino acid substitution mutations in codons 12, 13 or 61. These Ras mutations are found in up to approximately 6.5% of breast cancers, 30% of non-small cell lung cancers, 50% of colon cancers, and 100% of pancreatic cancers. Even in the absence of these mutations the Ras signaling pathway may be central to cancer development and progression, since several Ras pathway proteins upstream (e.g. epidermal growth factor receptor and Her2/neu) and downstream (e.g. Akt, ERK kinase) of Ras are also found to be amplified or mutated in human tumors. For example, although Ras is rarely found in mutated form in breast tumors, Ras overexpression and amplification has been observed in 50-70% of breast adenocarcinomas.

Complementary findings also support the role of ChoK in the generation of human tumors. For example, nuclear magnetic resonance (NMR) techniques have shown the presence of high PCho levels in several human tumor tissues including breast, prostate, brain and ovarian tumors with respect to normal tissues. ChoK appears to be activated by multiple growth factors and signal transducers that may be regulators of neoplastic growth and survival and may be implicated in the initiation and progression of human cancers.

Evidence for choline kinase activity in cancer has also been obtained from the observation that siRNA silencing of choline kinase mRNA expression by MDA-MB-231 breast adenocarcinoma cells reduces intracellular phosphocholine, which in turn decreases cellular proliferation and promotes differentiation. Although these studies were not conducted in vivo, they nevertheless support the validity of choline kinase as a molecular target for the development of anti-breast cancer agents.

Ras is one of the most intensely studied oncogenes in human carcinogenesis and ChoK inhibition has been hypothesized as an anti-tumor strategy with some success. The design of compounds directly affecting ChoK activity or the enzyme activated by phosphorylcholine has provided agents with anti-tumor effects in cells transformed by oncogenes, however the specific test drugs available to-date suffer from delivery and/or safety deficiencies which make them unsuitable for clinical use:

Several ChoK inhibitors are well-known in the art. Researchers identified Hemicholinium-3 (HC-3) as a relatively potent and selective blocking agent (Cuadrado A., et al., 1993, Oncogene 8: 2959-2968, e.g.). HC-3 is a choline homologue with a biphenyl structure and has been used for designing new anti-tumor drugs. However, HC-3 is a potent respiratory paralyzing agent and is therefore not a good candidate for use in clinical practice. Introduction of structural modifications have reduced toxic side effects but full retention of inhibitory activity is not achieved. Bisquaternized symmetric compounds derived from pyridinium have also been found to inhibit PCho production in whole cells (WO98/05644). However, these derivatives have high toxicity levels limiting extended therapeutic application. ChoK-specific siRNAs have been developed but use of an siRNA is not feasible due to a lack of suitable technology for transporting the siRNA to the tumor cell, and due to lack of selectivity of inhibition among ChoK isoforms. Mori et al (Cancer Res., 2007, 67:11284-11290).

Hence, there remains a need in the art for pharmaceutical compounds which effectively inhibit ChoK-alpha while reducing the toxic side effects which accompany the current state-of-the-art.

### SUMMARY

Choline kinase is a valuable molecular target for the development of anti-cancer therapeutics. Accordingly, provided herein are novel small molecule choline kinase inhibitors having the following formula:

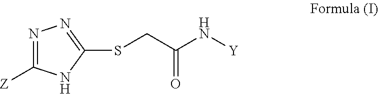

In another embodiment, a pharmaceutical composition for the treatment of cancer is provided, the composition comprising a Formula I compound and at least one pharmaceutically acceptable carrier.

In another embodiment, a method of treating cancer is provided, the method comprising administering to a subject in need thereof an effective amount of a Formula I compound.

In another embodiment, a method of inhibiting enzymatic activity of choline kinase in a cell is provided, the method comprising administering an effective amount of a Formula I compound.

In still another embodiment, a method of treating a tumor is provided, the method comprising administering to a subject in need thereof an effective amount of one or more Formula I compounds.

These and other objects, features, embodiments, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified.

### BRIEF DESCRIPTION OF THE DRAWINGS

FIG. 1 shows the time vs. plasma pharmacokinetic concentration profile of ACT-CK-50 in BalbC mice.

FIG. 2 shows the time vs. concentration pharmacokinetic profile of ACT-CK-154 in BalbC mice.

FIG. 3 shows the average tumor volume as a function of time for control group and treatment group (ACT-CK-50).

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

Throughout the specification and claims, a given chemical formula or name shall encompass all tautomers and optical and stereoisomers, as well as racemic mixtures where such isomers and mixtures exist.

The term “cancer” as used herein refers to diseases caused by uncontrolled cell division and the ability of cells to metastasize, or to establish new growth in additional sites. The terms “malignant,” “malignancy,” “neoplasm,” “tumor” and variations thereof refer to cancerous cells or groups of cancerous cells.

The term “anti-cancer agent,” “anti-cancer compound,” “anti-neoplastic compound,” “anti-tumor agent,” “anti-cancer therapeutic” and variations of as used herein refer to compounds that can prevent the proliferation of cancer cells and tumors or kill cancer cells.

Specific types of cancer include, but are not limited to, skin cancers, connective tissue cancers, adipose cancers, breast cancers, lung cancers, stomach cancers, pancreatic cancers, ovarian cancers, cervical cancers, uterine cancers, anogenital cancers, kidney cancers, bladder cancers, colon cancers, prostate cancers, central nervous system (CNS) cancers, retinal cancer, blood, and lymphoid cancers.

The term “competitive inhibitor” refers to an inhibitor whose binding to an enzyme prevents the binding of the enzyme's normal substrate.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

As used herein, the term “aza” refers to a heterocyclic ring structure containing at least one nitrogen atom. Specific examples of aza groups include, but are not limited to, pyrrolidine, piperidine, quinuclidine, pyridine, pyrrole, indole, purine, pyridazine, pyrimidine, and pyrazine.

The term “azaaryl” refers to a heterocyclic aryl group wherein one or more of the atoms of the aryl group ring or rings is nitrogen. Examples of azaaryl groups include monocyclic or bicyclic mono- or diazaaryl (i.e., an aryl group comprising two nitrogen atoms), which is unsubstituted or substituted by a member selected from the group consisting of lower alkyl, for example methyl, lower alkoxy, for example methoxy, and/or halogen, for example chlorine or bromine. Therefore, the term “azaaryl” refers to groups including, but not limited to, pyridine, pyridazine, pyrimidine, pyrazine, quinoline, quinaldine, quinoxaline, and substituted analogs thereof. In some embodiments, the azaaryl group is pyridyl, for example 2-, 3- or 4-pyridyl; quinolinyl or isoquinolinyl, for example 4-quinolinyl or 1-isoquinolinyl; imidazolyl; pyrimidinyl, for example 2- or 4-pyrimidinyl; pyridazinyl, for example 3-pyridazinyl; or pyrazinyl, for example 2-pyrazinyl.

A structure represented generally by a formula such as:

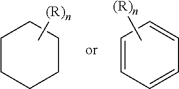

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

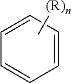

wherein n is an integer from 0 to 2 comprises compound groups including, but not limited to:

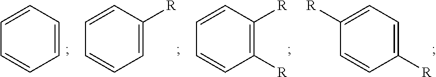

and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents,” including hydroxyl, halo, nitro, alkyl, aryl, aralkyl, carboxyl and the like. There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—(CH₂—O—); and ethylenedioxyl (—O—(CH₂)₂—O—). An alkylene group can have about 2 to about 3 carbon atoms and can further have 6-20 carbons.

As used herein, the term “acyl” refers to an organic carboxylic acid group wherein the —OH of the carboxyl group has been replaced with another substituent (i.e., as represented by RCO—, wherein R is an alkyl, aralkyl or aryl group as defined herein, including substituted alkyl, aralkyl, and aryl groups). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangeably with “alkoxyl.”

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NR— group wherein acyl is as previously described and R is H or alkyl. Thus, the “acylamino” group can have the structure —NR—C(═O)—R′, wherein R′ is alkyl, aryl, aralkyl, and the like.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

When the term “independently selected” is used, the substituents being referred to (e.g., R groups, such as groups R₂ and R₃, or groups B and C), can be identical or different. For example, both R₂ and R₃ can be the same substituent, or R₂ and R₃ can each be different substituents selected from a specified group.

Choline kinase commits choline to the cytidylyl diphosphate-choline (or Kennedy) pathway for the biosynthesis of phosphatidylcholine, the major phospholipid constituent in membranes. Choline kinase appears to be a rate-limiting enzyme for this pathway, as activation of choline kinase causes a corresponding increase in the rate of phosphatidylcholine synthesis. Neoplastic cells have a great need for membrane phospholipids as a result of both rapid cell proliferation and an increased rate of endosome formation required for growth factor signaling and the secretion of microvesicles or exosomes.

Thus, selectively targeting the family of choline kinase enzymes, more specifically choline kinase alpha, with small molecule inhibitors is a new strategy for cancer therapy.

U.S. application Ser. No. 12/824,680, filed Jun. 18, 2010 and incorporated herein by reference in its entirety, disclosed certain small molecule inhibitors of choline kinase. Surprisingly, the instant inventors have discovered that the presently disclosed compounds having ethoxy and other moieties at the Z position of Formula I exhibit a significantly higher in vitro cytotoxicity profile, as illustrated herein by Examples 1 and 5.

The compounds of the present invention are novel anti-cancer compounds that inhibit the enzymes of the choline kinase family, more specifically choline kinase alpha.

In some embodiments, the compounds of the present invention describes a compound that inhibits or otherwise changes the activity of choline kinase alpha, wherein the compound is a compound of Formula I described above.

### I. Compounds

The choline kinase inhibitors of the present invention have the following structural formula:

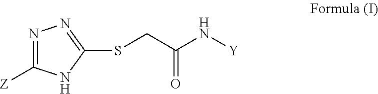

wherein:

Z is selected from the group consisting of:

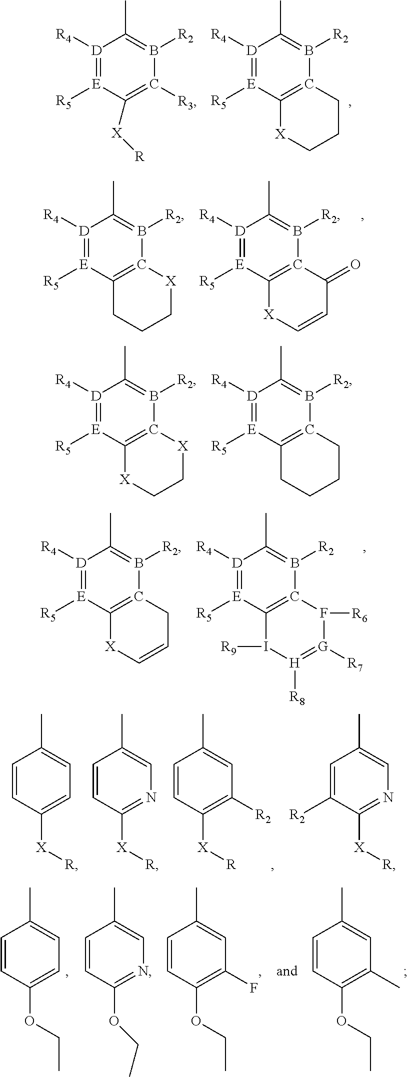

Y is selected from the group consisting of:

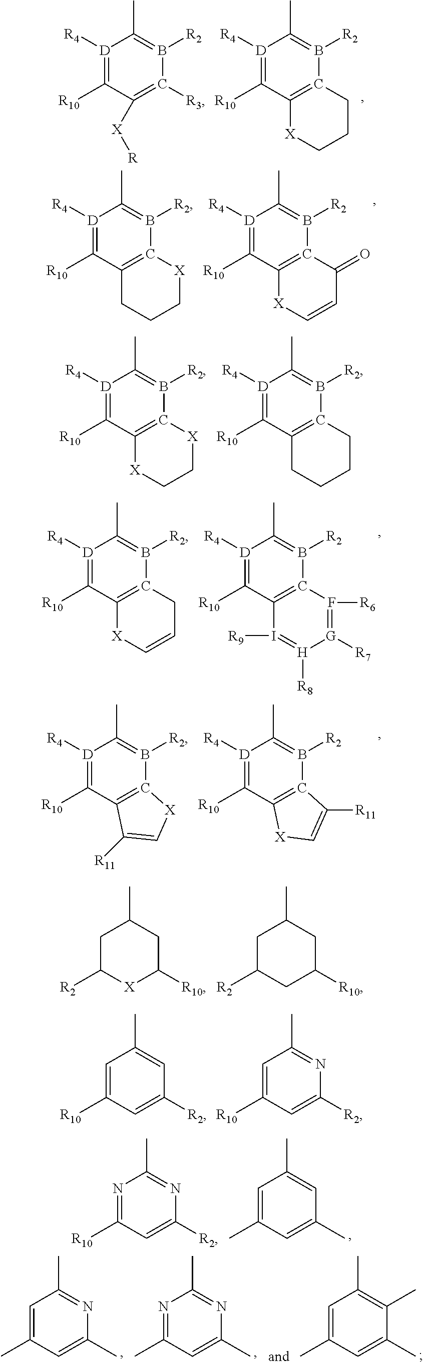

X is O, S, NH, or N-alkyl;

R is alkyl or substituted alkyl;

B, C, D, E, F, G, H, and I are independently selected from the group consisting of N or C substituted with one of R₂, R₃, R₄, R₅, R₆, R₇, R₈, or R₉, wherein if B, C, D, E, F, G, H, or I are N, then R₂, R₃, R₄, R₅, R₆, R₇, R₈, or R₉ represent the free electron pair at the N atom;

R₂, R₃, R₄, R₅, R₆, R₇, R₈, or R₉ are independently, when attached to an N atom, a free electron pair, or, when attached to C, selected from the group consisting of hydrogen, halogen, cyano, nitro, straight-chain or branched (C₁-C₆)-alkyl, straight-chain or branched (C₁-C₆)-alkyl substituted with one or more halogen atoms, straight-chain or branched (C₁-C₆)-alkoxy substituted with one or more halogen atoms, (C₂-C₆)-alkenyl, (C₂-C₆)-alkynyl, (C₃-C₈)-cycloalkyl, straight-chain or branched (C₁-C₆)-alkoxy, straight-chain or branched (C₁-C₆)-alkylenedioxy, (C₁-C₆)-alkoxycarbonyl, (C₁-C₆)-alkoxycarbonyloxy, (C₁-C₆)-alkylcarbonyl, (C₁-C₆)-alkylcarbonyloxy, (C₁-C₆)-alkylthio, (C₁-C₆)-alkylsulfinyl, (C₁-C₆)-alkylsulfonyl, carboxyl, (C₁-C₆)-alkyl carboxylate, carboxamide, N—(C₁-C₄)-alkyl-carboxamide, N,N-di-(C₁-C₄)-alkyl-carboxamide, (C₁-C₆)-alkoxy-(C₁-C₆)-alkyl, amino, mono-(C₁-C₆)-alkylamino, N,N-di-(C₁-C₆)-alkylamino, where two C₁-C₆-alkyl radicals together may form a ring, which optionally contains one or more of NH, N—(C₁-C₆)-alkyl, O or S, (C₆-C₁₄)-aryl, (C₆-C₁₄)-aryloxy, (C₆-C₁₄)-aryl-(C₁-C₄)-alkyl, (C₆-C₁₄)-aryl-(C₁-C₄)-alkoxy-(C₁-C₄)-alkyl, (C₁-C₆)-alkylcarbonyl, (C₁-C₆)-alkoxycarbonyl, hydroxyl, wherein two directly adjacent radicals may be attached to one another; and

R₁₀ is alkyl or substituted alkyl.

Where prototropic hydrogens exist on the heterocycle of Formula I, structural depictions are understood to include the tautomeric forms (e.g., 1H, 2H, and 4H tautomeric forms thereof):

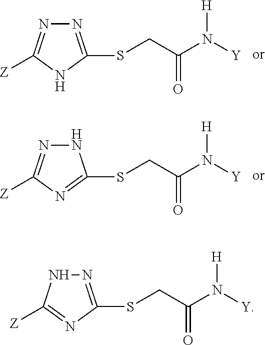

Examples of Formula I compounds are shown in Table 1, below.

TABLE 1
Exemplary Formula I Compounds

| Compound | | | |
|---|---|---|---|
| Number | MW | Name & NMR Data | Structure |
| ACT-CK- 050 | 382.5 | N-(3,5-Dimethyl-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.34 (t, J = 6.9 Hz, 3H), 2.22 (s, 6H), 4.05-4.16 (m, 4H), 6.70 (s, 1H), 7.00-7.05 (m, 2H), 7.20 (s, 2H), 7.87 (d, J = 9.0 Hz, 2H), 10.11 (s, 1H), 14.27 (s, 1H). | 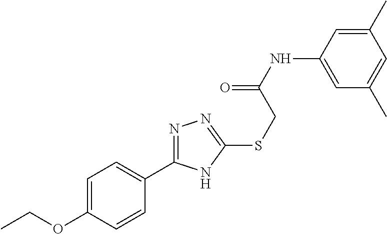 |
| ACT-CK- 060 | 264.3 | N-(2,6-Difluoro-phenyl)-2-[5-(4-fluoro-phenyl)- 4H-[1,2,4]triazol-3-ylsulfanyl]-acetamide. It was prepared using Scheme-1 as white solid. 1H NMR (300 MHz, DMSO-d₆) δ 3.98 (s, 2H), 6.97-6.99 (m, 2H), 7.11-7.25 (m, 3H), 8.00-8.05 (m, 2H), 9.80 (s, 1H), 14.31 (s, 1H). | 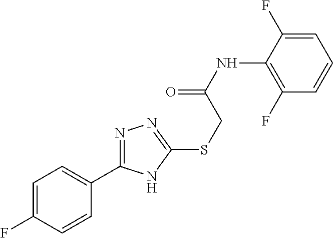 |
| ACT-CK- 061 | 376.4 | N-(2,6-Difluoro-phenyl)-2-[5-(4-methoxy-phenyl)- 4H-[1,2,4]triazol-3-ylsulfanyl]-acetamide. It was prepared using Scheme-2 as white solid. 1H NMR (300 MHz, DMSO-d₆) δ 3.85 (s, 3H), 3.95 (s, 2H), 6.93-6.97 (m, 4H), 7.12-7.22 (m, 1H), 7.94 (d, J = 9.0 Hz, 2H), 10.06 (s, 1H), 13.90 (s, 1H). | 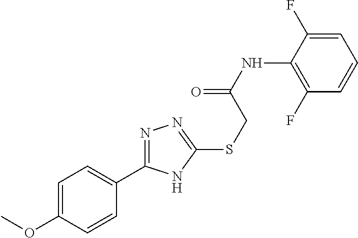 |
| ACT-CK- 063 | 376.4 | N-(2,6-Difluoro-phenyl)-2-[5-(4-methoxy-phenyl)- 4H-[1,2,4]triazol-3-ylsulfanyl]-acetamide. It was prepared using Scheme-2 as white solid. 1H NMR (300 MHz, DMSO-d₆) δ 3.86 (d, J = 5.1 Hz, 3H), 3.93 (s, 2H), 6.82-6.89 (m, 2H), 6.98 (d, J = 8.7 Hz, 2H), 7.97 (d, J = 8.1 Hz, 2H), 8.21-8.26 (m, 1H), 10.11 (s, 1H), 14.09 (br s, 1H). | 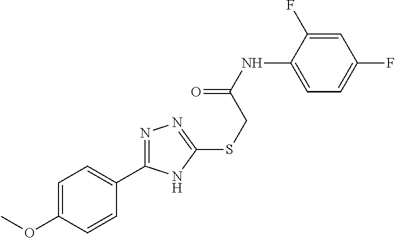 |
| ACT-CK- 064 | 390.4 | N-(2,4-Difluoro-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.34 (t, J = 6.9 Hz, 3H), 4.05-4.11 (m, 4H), 6.96-7.10 (m, 3H), 7.29-7.36 (m, 1H), 7.82-7.89 (m, 3H), 10.08 (s, 1H), 14.31 (br s, 1H). | 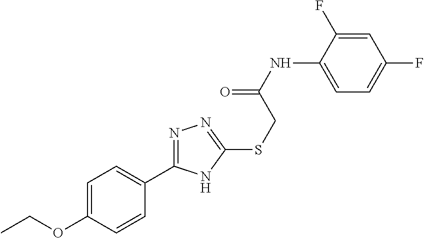 |
| ACT-CK- 066 | 372.4 | N-(5-Fluoro-2-methyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, CDCl₃) δ 2.06 (s, 3H), 3.85 (s, 3H), 3.96 (s, 2H), 6.70-6.74 (m, 1H), 6.94-7.04 (m, 3H), 7.77-7.81 (m, 1H), 7.89 (s, 1H), 7.93 (s, 1H), 9.44 (s, 1H), 13.99 (br s, 1H). | 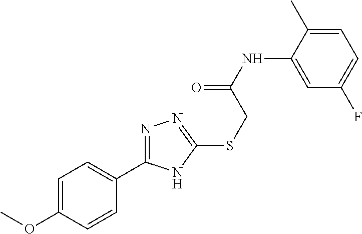 |
| ACT-CK- 067 | 386.4 | 2-[5-(4-Ethoxy-phenyl)-4H-[1,2,4]triazol-3- ylsulfanyl]-N-(5-fluoro-2-methyl-phenyl)- acetamide. It was prepared using Scheme-2 as white solid. ¹H NMR (300 MHz, DMSO-d₆) δ 1.34 (t, J = 6.9 Hz, 3H), 2.15 (s, 3H), 4.05-4.13 (m, 4H), 6.88-6.94 (m, 1H), 7.04 (s, 1H), 7.06 (s, 1H), 7.19-7.24 (m, 1H), 7.42-7.47 (m, 1H), 7.87 (s, 1H), 7.90 (s, 1H), 9.62 (s, 1H), 14.31 (br s, 1H). | 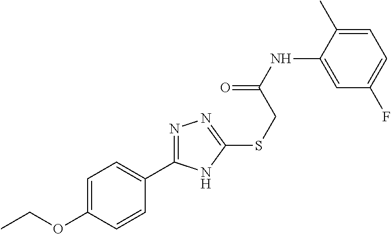 |
| ACT-CK- 070 | 368.5 | N-(2,3-Dimethyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, CDCl₃) δ 2.02 (s, 3H), 2.23 (s, 3H), 3.84 (s, 3H), 3.95 (s, 2H), 6.93-6.98 (m, 3H), 7.06 (t, J = 7.8 Hz, 1H), 7.45-7.48 (m, 1H), 7.90 (s, 1H), 7.93 (s, 1H), 9.39 (s, 1H), 14.05 (br s, 1H). | 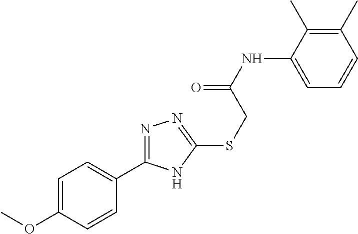 |
| ACT-CK- 071 | 386.4 | 2-[5-(4-Ethoxy-phenyl)-4H-[1,2,4]triazol-3- ylsulfanyl]-N-(2-fluoro-5-methyl-phenyl)- acetamide. It was prepared using Scheme-2 as white solid. ¹H NMR (300 MHz, DMSO-d₆) δ 1.34 (t, J = 6.9 Hz, 3H), 2.25 (s, 3H), 3.92-4.14 (m, 4H), 6.89-7.17 (m, 4H), 7.69-7.86 (m, 3H), 9.98 (s, 1H), 14.31 (br s, 1H). | 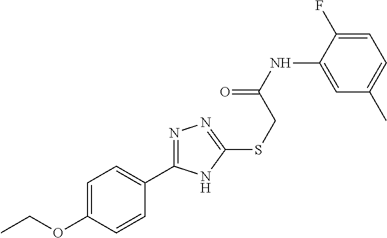 |
| ACT-CK- 076 | 382.5 | N-(2,3-Dimethyl-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.35 (t, J = 6.9 Hz, 3H), 2.04 (s, 3H), 2.22 (s, 2H), 4.05-4.12 (m, 4H), 6.99-7.07 (m, 4H), 7.88 (s, 1H), 7.91 (s, 1H), 9.66 (s, 1H), 14.26 (br.s, 1H). | 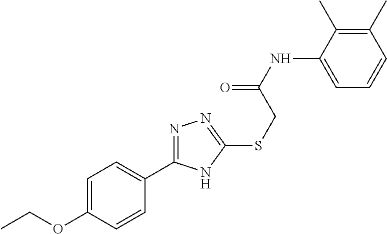 |
| ACT-CK- 079 | 384.5 | N-(5-Methoxy-2-methyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 2.11 (s, 3H), 3.71 (s, 3H), 3.82 (s, 3H), 4.05 (s, 2H), 6.71-6.77 (m, 2H), 7.00-7.11 (m, 2H), 7.22-7.25 (m, 1H), 7.89 (s, 1H), 7.92 (s, 1H), 9.43 (s, 1H), 14.32 (s, 1H). | 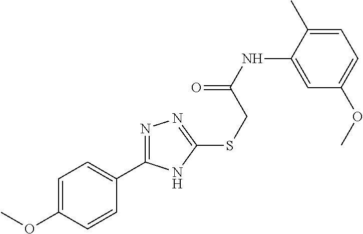 |
| ACT-CK- 082 | 376.4 | N-(3,5-Difluoro-phenyl)-2-[5-(4-methoxy-phenyl)- 4H-[1,2,4]triazol-3-ylsulfanyl]-acetamide. It was prepared using Scheme-2 as white solid. 1H NMR (300 MHz, DMSO-d₆) δ 3.81 (s, 3H), 4.11 (s, 2H), 6.93 (tt, J = 9.3 Hz, 2.4 Hz, 1H), 7.06 (m, 2H), 7.29-7.33 (m, 2H), 785-7.89 (m, 2H), 10.70 (s, 1H), 14.21 (br s, 1H). | 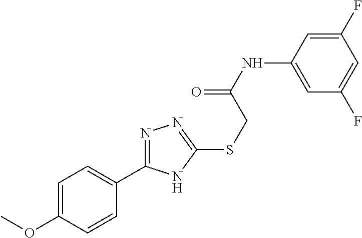 |
| ACT-CK- 083 | 390.4 | N-(3,5-Difluoro-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.34 (t, J = 6.9 Hz, 3H), 4.05-4.11 (m, 4H), 6.93-6.97 (m, 1H), 7.03 (s, 1H), 7.05 (s, 1H), 7.32 (m, 2H), 7.86 (m, 2H), 10.69 (s, 1H), 14.26 (br. s, 1H). | 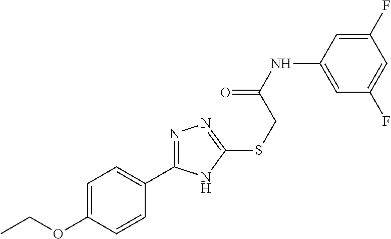 |
| ACT-CK- 085 | 372.4 | N-(2-Fluoro-5-methyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 2.25 (s, 3H), 3.82 (s, 3H), 4.13 (s, 2H), 6.92-6.95 (m, 1H), 7.06-7.16 (m, 1H), 7.75 (d, J = 7.2 Hz, 1H), 7.89 (s, 1H), 7.91 (s, 1H), 10.01 (s, 1H), 14.32 (br s, 1H). | 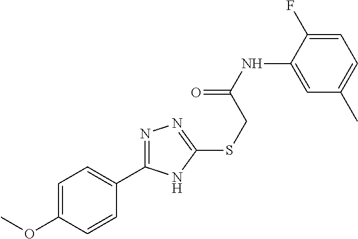 |
| ACT-CK- 087 | 398.5 | 2-[5-(4-Ethoxy-phenyl)-4H-[1,2,4]triazol-3- ylsulfanyl]-N-(4-methoxy-2-methyl-phenyl)- acetamide. It was prepared using Scheme-2 as white solid. ¹H NMR (300 MHz, DMSO-d₆) δ 1.35 (t, J = 6.9 Hz, 3H), 2.11 (s, 3H), 3.71 (s, 3H), 4.05-4.12 (m, 4H), 6.70-6.78 (m, 2H), 7.04 (s, 1H), 7.07 (s, 1H), 7.23 (d, J = 8.7 Hz, 1H), 7.86 (m, 2H), 7.88 (s, 1H), 7.91 (s, 1H), 9.49 (s, 1H), 14.25 (br. s, 1H). | 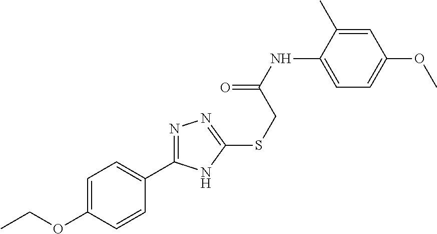 |
| ACT-CK- 089 | 372.4 | N-(4-Fluoro-3-methyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 2.18 (s, 3H), 3.79 (s, 3H), 4.05 (s, 2H), 7.03-7.09 (m, 3H), 7.34-7.39 (m, 1H), 7.48 (dd, J = 6.9 Hz, 2.1 Hz, 1H), 7.85 (s, 1H), 7.88 (s, 1H), 10.25 (s, 1H), 14.21 (br s, 1H). | 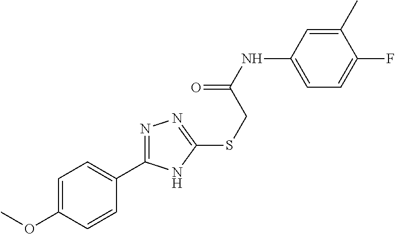 |
| ACT-CK- 090 | 386.4 | 2-[5-(4-Ethoxy-phenyl)-4H-[1,2,4]triazol-3- ylsulfanyl]-N-(4-fluoro-3-methyl-phenyl)- acetamide. It was prepared using Scheme-2 as white solid. ¹H NMR (300 MHz, DMSO-d₆) δ 1.35-1.40 (m, 3H), 2.20 (s, 3H), 4.05-4.12 (m, 4H), 7.03-7.11 (m, 3H), 7.37-7.42 (m, 1H), 7.49-7.52 (m, 1H), 7.85-7.89 (m, 2H), 10.30 (s, 1H). | 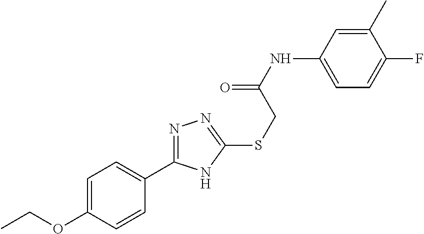 |
| ACT-CK- 092 | 400.5 | N-(2,5-Dimethoxy-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 3.67 (s, 6H), 3.82 (s, 3H), 4.09-4.21 (m, 2H), 6.61 (d, J = 8.1 Hz, 1H), 6.90-7.11 (m, 3H), 7.75-7.93 (m, 3H), 9.50 (s, 1H), 14.38 (br s, 1H). | 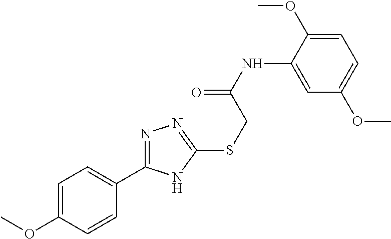 |
| ACT-CK- 093 | 414.5 | N-(2,5-Dimethoxy-phenyl)-2-[5-(4-ethoxy- phenyl)-4H-[1,2,4]triazol-3-ylsulfanyl]-acetamide. It was prepared using Scheme-2 as Light orange solid; ¹H NMR (300 MHz, DMSO-d₆) δ 1.35 (t, J = 6.9 Hz, 3H), 3.66 (s, 6H), 4.06-4.12 (m, 4H), 6.60-6-63 (m, 1H), 6.92 (d, J = 9 Hz, 1H), 7.06-7.08 (m, 2H), 7.80 (s, 2H), 7.89-7.92 (m, 2H), 9.50 (s, 1H), 14.36 (br s, 1H). | 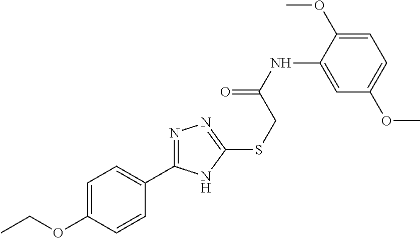 |
| ACT-CK- 096 | 368.5 | N-(2,4-Dimethoxy-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 2.11 (s, 3H), 2.23 (s, 3H), 3.82 (s, 2H), 4.09 (s, 2H), 6.94-7.00 (m, 2H), 7-06-7.09 (m, 2H), 7.28 (d, J = 8.1 Hz, 1H), 7.90-7.93 (m, 2H), 9.52 (s, 1H), 14.27 (br s, 1H). | 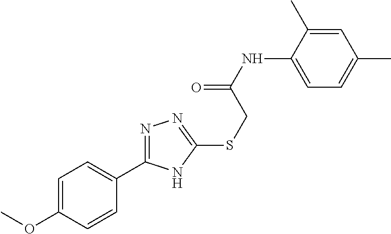 |
| ACT-CK- 097 | 382.5 | N-(2,4-Dimethyl-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.35 (t, J = 6.9 Hz, 3H), 2.11 (s, 3H), 2.23 (s, 3H), 4.05-4.12 (m, 4H), 6.94-7.07 (m, 4H), 7.29 (d, J = 8.1 Hz, 1H), 7.88-7.91 (m, 2H), 9.52 (s, 1H), 14.25 (br s, 1H). | 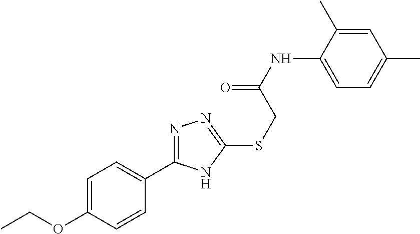 |
| ACT-CK- 099 | 361.4 | 2-[5-(4-Methoxy-phenyl)-4H-[1,2,4]triazol-3- ylsulfanyl]-N-(5-methyl-thiazol-2-yl)-acetamide. It was prepared using Scheme-2 as white solid. 1H NMR (300 MHz, DMSO-d₆) δ 2.33 (s, 3H), 3.81 (s, 3H), 4.15 (s, 2H), 7.04-7.15 (m, 3H), 7.85-7.88 (m, 2H), 12.21 (br s, 1H), 14.28 (br s, 1H). | 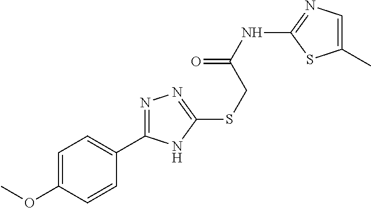 |
| ACT-CK- 100 | 375.5 | 2-[5-(4-Ethoxy-phenyl)-4H-[1,2,4]triazol-3- ylsulfanyl]-N-(5-methyl-thiazol-2-yl)-acetamide.. It was prepared using Scheme-2 as white solid. ¹H NMR (300 MHz, DMSO-d₆) δ 1.32-1.36 (m, 3H), 2.33 (s, 3H), 4.05-4.14 (m, 4H), 7.03-7.06 (m, 2H), 7.14 (s, 1H), 7.84-7.87 (m, 2H), 12.21 (s, 1H), 14.31 (br s, 1H). | 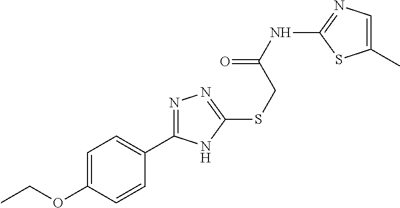 |
| ACT-CK- 102 | 372.4 | N-(2-Fluoro-4-methyl-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 2.27 (s, 3H), 3.82 (s, 3H), 4.13 (s, 2H), 6.96 (d, J = 8.1 Hz, 1H), 7.06-7.10 (m, 3H), 7.76 (t, J = 8.4 Hz, 1H), 7.89-7.92 (m, 2H), 9.99 (s, 1H), 14.32 (br s, 1H). | 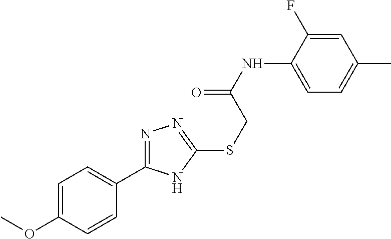 |
| ACT-CK- 103 | 386.4 | 2-[5-(4-Ethoxy-phenyl)-4H-[1,2,4]triazol-3- ylsulfanyl]-N-(2-fluoro-4-methyl-phenyl)- acetamide. was prepared using Scheme-2 as white solid. ¹H NMR (300 MHz, DMSO-d₆) δ 1.35 (t, J = 6.9 Hz, 3H), 2.27 (s, 3H), 4.05-4.12 (m, 4H), 6.96 (d, J = 8.4 Hz, 1H), 7.04-7.10 (m, 3H), 7.74-7.80 (m, 1H), 7.87-7.90 (m, 2H), 9.98 (s, 1H), 14.29 (br s, 1H). | 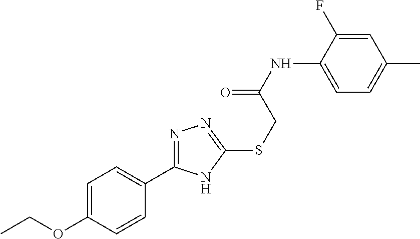 |
| ACT-CK- 105 | 442.8 | N-(4-Chloro-3-trifluoromethyl-phenyl)-2-[5-(4- methoxy-phenyl)-4H-[1,2,4]triazol-3-ylsulfanyl]- acetamide. It was prepared using Scheme-2 as white solid. 1H NMR (300 MHz, DMSO-d₆) δ 3.82 (s, 3H), 4.12 (s, 2H), 7.04-7.07 (m, 2H) 7.68 (d, J = 8.7 Hz, 1H), 7.83-7.90 (m, 3H), 8.19 (s, 1H), 10.76 (s, 1H), 14.24 (br s, 1H). | 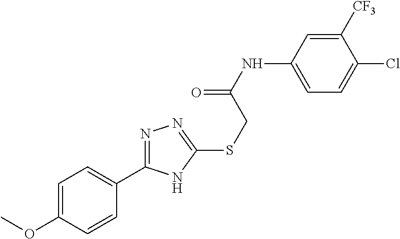 |
| ACT-CK- 106 | 456.9 | N-(4-Chloro-3-trifluoromethyl-phenyl)-2-[5-(4- ethoxy-phenyl)-4H-[1,2,4]triazol-3-ylsulfanyl]- acetamide. It was prepared using Scheme-2 as white solid. ¹H NMR (300 MHz, DMSO-d₆) δ 1.35 (t, J = 6.9 Hz, 3H) 4.05-4.12 (m, 4H), 7.03-7.06 (m, 2H), 7.68 (d, J = 8.7 Hz, 1H), 7.83-7.88 (m, 3H), 8.20 (s, 1H), 10.17 (s, 1H), 14.31 (s, 1H). | 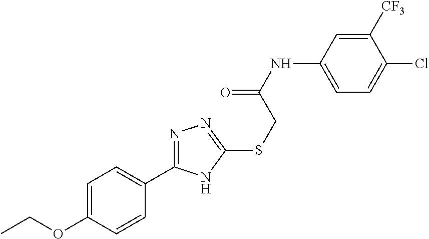 |
| ACT-CK- 108 | 390.4 | N-(2,3-Difluoro-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.34 (t, J = 6.9 Hz, 3H), 4.08-4.15 (m, 4H), 6.95-7.07 (m, 3H), 7.29-7.34 (m, 1H), 7.86-7.89 (m, 3H), 10.26 (br s, 1H), 14.32 (br s, 1H). | 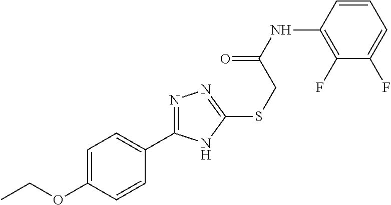 |
| ACT-CK- 109 | 414.5 | N-(3,5-Dimethoxy-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.32-1.37 (m, 3H), 3.69 (s, 6H), 4.05-4.12 (m, 4H), 6.22 (s, 1H), 6.83 (s, 2H), 7.05-7.07 (m, 2H), 7.86-7.87 (m, 2H), 10.24 (s, 1H), 14.29 (s, 1H). | 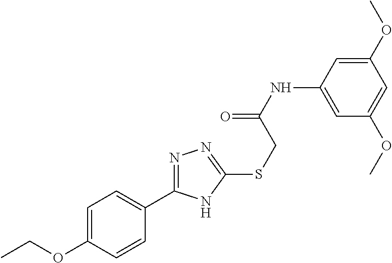 |
| ACT-CK- 110 | 400.5 | N-(3,5-Dimethoxy-phenyl)-2-[5-(4-methoxy- phenyl)-4H-[1,2,4]triazol-3-ylsulfanyl]-acetamide. It was prepared using Scheme-2 as white solid. 1H NMR (300 MHz, DMSO-d₆) δ 3.37 (s, 6H), 3.70 (s, 3H), 4.06 (s, 2H), 6.23 (s, 1H), 6.84 (s, 2H), 7.07-7.09 (m, 2H), 7.88 (s, 1H), 7.91 (s, 1H), 10.25 (s, 1H), 14.31 (s, 1H). | 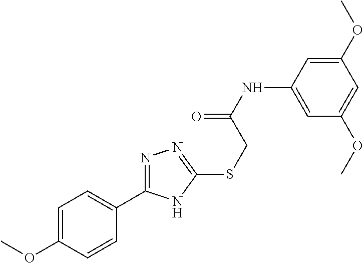 |
| ACT-CK- 111 | 376.4 | N-(2,5-Difluoro-phenyl)-2-[5-(4-methoxy-phenyl)- 4H-[1,2,4]triazol-3-ylsulfanyl]-acetamide. It was prepared using Scheme-2 as white solid. 1H NMR (300 MHz, DMSO-d₆) δ 3.82 (s, 3H), 4.17 (s, 2H), 7.05-7.08 (m, 2H), 7.17 (t, J = 6.6 Hz, 2H), 7.72 (t, J = 7.2 Hz, 1H), 7.88 (s, 1H), 7.91 (s, 1H), 10.29 (s, 1H), 14.31 (br s, 1H). | 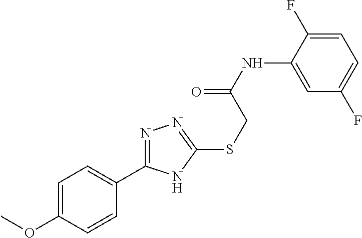 |
| ACT-CK- 112 | 376.4 | N-(2,3-Difluoro-phenyl)-2-[5-(4-methoxy-phenyl)- 4H-[1,2,4]triazol-3-ylsulfanyl]-acetamide. It was prepared using Scheme-2 as white solid. 1H NMR (300 MHz, DMSO-d₆) δ 3.82 (s, 3H), 4.17 (s, 2H), 7.05-7.08 (m, 2H), 7.17 (t, J = 6.6 Hz, 2H), 7.72 (t, J = 7.2 Hz, 1H), 7.88 (s, 1H), 7.91 (s, 1H), 10.29 (s, 1H), 14.31 (br s, 1H). | 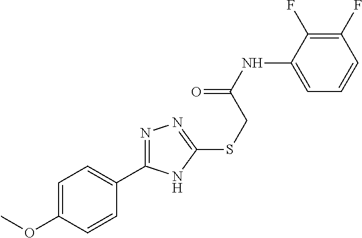 |
| ACT-CK- 113 | 390.4 | N-(2,5-Difluoro-phenyl)-2-[5-(4-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.35 (t, J = 6.9 Hz, 3H) 4.05-4.17 (m, 4H), 6.93-7.06 (m, 3H), 7.28-7.37 (m, 1H), 7.85-7.93 (m, 3H), 10.27 (s, 1H), 14.28 (br s, 1H). | 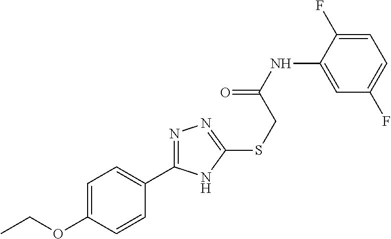 |
| ACT-CK- 114 | 404.4 | N-(2,3-Difluoro-phenyl)-2-[5-(4-ethoxy-phenyl)-4- methyl-4H-[1,2,4]triazol-3-ylsulfanyl]-acetamide. It was prepared using Scheme-2 as white solid. ¹H NMR (300 MHz, DMSO-d₆) δ 1.36 (t, J = 6.9 Hz, 3H), 3.62 (s, 3H), 4.11 (q, J = 6.9 Hz, 2H), 4.19 (s, 2H), 6.98-7.02 (m, 1H), 7.07-7.11 (m, 2H), 7.29-7.37 (m, 1H), 7.59-7.63 (m, 2H), 7.86-7.89 (m, 1H), 10.37 (s, 1H). | 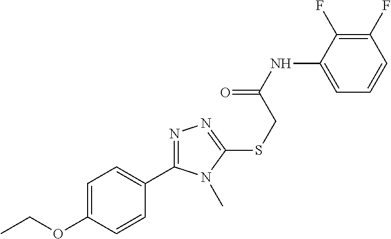 |
| ACT-CK- 115 | 400.5 | 2-[5-(4-Ethoxy-phenyl)-4-methyl-4H- [1,2,4]triazol-3-ylsulfanyl]-N-(2-fluoro-5-methyl- phenyl)-acetamide. It was prepared using Scheme-2 as white solid. ¹H NMR (300 MHz, DMSO-d₆) δ 1.36 (t, J = 6.9 Hz, 3H), 2.25 (s, 3H), 3.61 (s, 3H), 4.07-4.15 (m, 4H), 6.94-6.97 (m, 1H), 7.07-7.16 (m, 3H), 7.60-7.72 (m, 3H), 10.08 (s, 1H). | 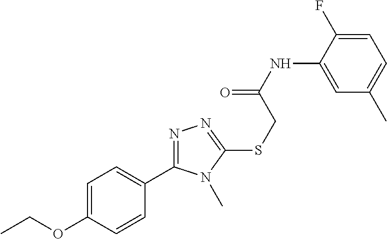 |
| ACT-CK- 116 | 396.5 | N-(3,5-Dimethyl-phenyl)-2-[5-(4-ethoxy-phenyl)-4- methyl-4H-[1,2,4]triazol-3-ylsulfanyl]-acetamide. It was prepared using Scheme-2 as white solid. ¹H NMR (300 MHz, DMSO-d₆) δ 1.36 (t, J = 6.9 Hz, 3H), 2.22 (s, 6H), 3.60 (s, 3H), 4.07-4.14 (m, 4H), 6.71 (s, 1H), 7.07-7.10 (m, 2H), 7.18 (s, 2H), 7.59-7.62 (m, 2H), 10.15 (s, 1H); | 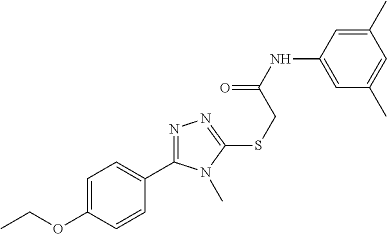 |
| ACT-CK- 117 | 404.4 | N-(2,5-Difluoro-phenyl)-2-[5-(4-ethoxy-phenyl)-4- methyl-4H-[1,2,4]triazol-3-ylsulfanyl]-acetamide. It was prepared using Scheme-2 as white solid. ¹H NMR (300 MHz, DMSO-d₆) δ 1.36 (t, J = 6.9 Hz, 3H), 3.62 (s, 3H), 4.07-4.19 (m, 4H), 6.99-7.10 (m, 3H), 7.29-7.37 (m, 1H), 7.60-7.66 (m, 2H), 7.89 (br s 1H), 10.37 (s, 1H). | 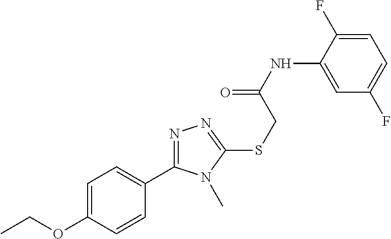 |
| ACT-CK- 118 | 396.5 | N-(3,5-Dimethyl-phenyl)-2-[5-(4-propoxy-phenyl)- 4H-[1,2,4]triazol-3-ylsulfanyl]-acetamide. It was prepared using Scheme-2 as white solid ¹H NMR (300 MHz, DMSO-d₆) δ 0.99 (t, J = 7.2 Hz, 3H), 1.71-1.78 (m, 2H), 2.22 (s, 6H), 3.96-4.06 (m, 4H), 6.70 (s, 1H), 7.05 (d, J = 8.4 Hz, 2H), 7.20 (s, 2H), 7.88 (d, J = 8.7 Hz, 2H), 10.13 (s, 1H), 14.26 (br s, 1H). | 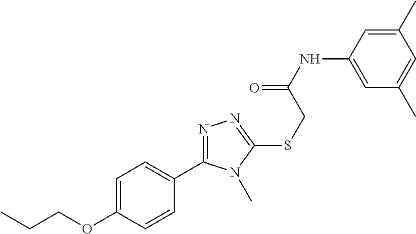 |
| ACT-CK- 119 | 428.5 | N-(3,5-Dimethoxy-phenyl)-2-[5-(4-propoxy- phenyl)-4H-[1,2,4]triazol-3-ylsulfanyl]-acetamide. It was prepared using Scheme-2 as white solid. ¹H NMR (300 MHz, DMSO-d₆) δ 0.99 (t, J = 7.2 Hz, 3H), 1.69-1.80 (m, 2H), 3.70 (s, 6H), 3.98 (t, J = 6.3 Hz, 2H), 4.07 (s, 2H), 6.21-6.23 (m, 1H), 6.83 (s, 2H), 7.05 (d, J = 8.7 Hz, 2H), 7.88 (d, J = 8.7 Hz, 2H), 10.26 (s, 1H), 14.23 (br s, 1H). | 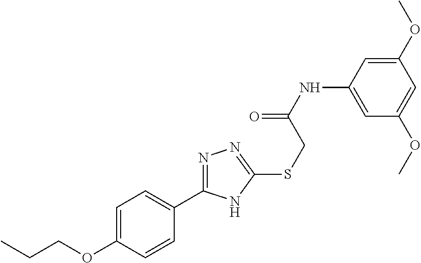 |
| ACT-CK- 120 | 400.5 | N-(2-Fluoro-5-methyl-phenyl)-2-[5-(4-propoxy- phenyl)-4H-[1,2,4]triazol-3-ylsulfanyl]-acetamide. It was prepared using Scheme-2 as white solid. ¹H NMR (300 MHz, DMSO-d₆) δ 0.99 (t, J = 7.2 Hz, 3H), 1.69-1.81 (m, 2H), 2.25 (s, 3H), 3.99 (t, J = 6.6 Hz, 2H), 4.13 (s, 2H), 6.94-6.96 (m, 1H), 7.04-7.16 (m, 3H), 7.75 (d, J = 6.9 Hz, 1H), 7.89 (d, J = 9.0 Hz, 2H), 10.00 (s, 1H), 14.28 (s, 1H). | 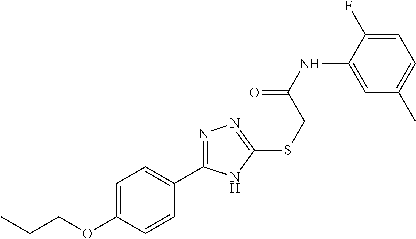 |
| ACT-CK- 124 | 377.5 | N-(3,5-Dimethyl-phenyl)-2-[5-(1H-indol-2-yl)-4H- [1,2,4]triazol-3-ylsulfanyl]-acetamide. It was prepared using Scheme-1 as white solid. 1H NMR (300 MHz, DMSO-d₆) δ 2.21 (s, 6H), 4.08 (s, 2H), 6.70 (s, 1H), 6.88-7.21 (m, 5H), 7.43-7.64 (m, 2H), 10.10 (s, 1H), 11.86 (s, 1H), 14.52 (br s, 1H). | 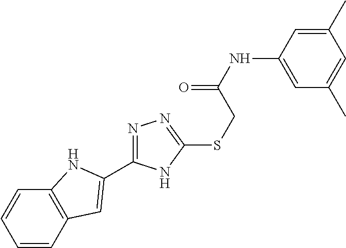 |
| ACT-CK- 125 | 394.5 | 2-[5-(4-Ethoxy-phenyl)-4H-[1,2,4]triazol-3- ylsulfanyl]-N-indan-5-yl-acetamide.. It was prepared using Scheme-2 as white solid. ¹H NMR (300 MHz, DMSO-d₆) δ 1.34 (t, J = 6.3 Hz, 3H), 1.97-1.99 (m, 2H), 2.79-2.81 (m, 4H), 4.07 (m, 4H), 7.06-7.29 (m, 4H), 7.50 (s, 1H), 7.86-7.88 (m, 2H), 10.16 (s, 1H), 14.27 (br s, 1H). | 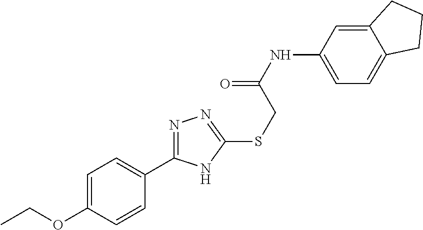 |
| ACT-CK- 126 | 383.5 | 2-[5-(4-Ethoxy-phenyl)-4H-[1,2,4]triazol-3- ylsulfanyl]-N-(6-ethyl-pyridin-2-yl)-acetamide. It was prepared using Scheme-2 as white solid. ¹H NMR (300 MHz, DMSO-d₆) δ 1.12-1.23 (m, 3H), 1.35 (t, J = 6.6 Hz, 3H), 2.64-2.72 (m, 2H), 4.07-4.10 (m, 4H), 7.00-7.05 (m, 3H), 7.66-7.71 (m, 1H), 7.86-7.93 (m, 3H), 10.78 (s, 1H), 14.27 (s, 1H). | 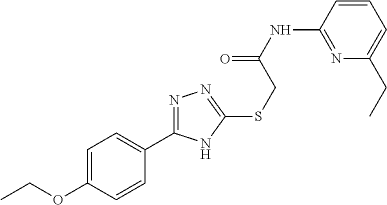 |
| ACT-CK- 127 | 410.5 | 2-[5-(4-Butoxy-phenyl)-4H-[1,2,4]triazol-3- ylsulfanyl]-N-(3,5-dimethyl-phenyl)-acetamide. It was prepared using Scheme-2 as white solid. ¹H NMR (300 MHz, DMSO-d₆) δ 0.94 (t, J = 7.5 Hz, 3H), 1.43-1.45 (m, 2H), 1.69-1.74 (m, 2H), 2.22 (s, 6H), 4.00-4.05 (m, 4H), 6.70 (s, 1H), 7.05-7.07 (m, 2H), 7.20 (s, 2H), 7.86-7.89 (m, 2H), 10.13 (s, 1H), 14.27 (s, 1H). | 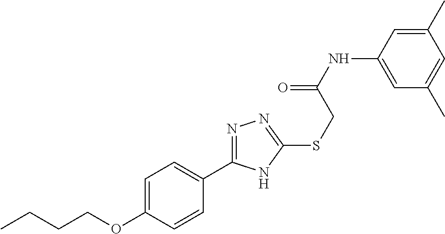 |
| ACT-CK- 128 | 414.5 | 2-[5-(4-Butoxy-phenyl)-4H-[1,2,4]triazol-3- ylsulfanyl]-N-(2-fluoro-5-methyl-phenyl)- acetamide. It was prepared using Scheme-2 as white solid. ¹H NMR (300 MHz, DMSO-d₆) δ 0.94 (t, J = 7.5 Hz, 3H), 1.41-1.48 (m, 2H), 1.67-1.74 (m, 2H), 2.25 (s, 3H), 4.03 (t, J = 6.3 Hz, 2H), 4.13 (s, 2H), 6.94-7.16 (m, 4H), 7.75 (t, J = 7.2 Hz, 1H), 7.87-7.89 (m, 2H), 10.01 (s, 1H), 14.26 (brs, 1H). | 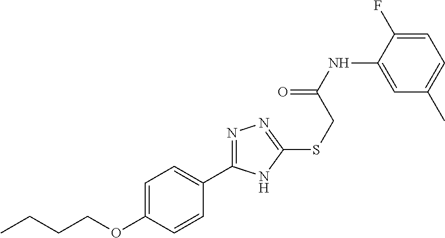 |
| ACT-CK- 129 | 396.5 | N-(3,5-Dimethyl-phenyl)-2-[5-(4-isopropoxy- phenyl)-4H-[1,2,4]triazol-3-ylsulfanyl]-acetamide. It was prepared using Scheme-2 as white solid. ¹H NMR (300 MHz, DMSO-d₆) δ 1.25-1.30 (m, 6H), 2.22 (s, 6H), 4.06 (s, 2H), 4.67-4.71 (m, 1H), 6.70 (s, 1H), 7.02-7.05 (m, 2H), 7.21 (s, 2H), 7.85-7.88 (m, 2H), 10.14 (s, 1H), 14.22 (brs, 1H). | 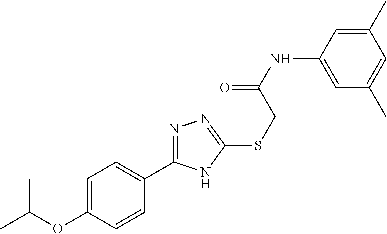 |
| ACT-CK- 130 | 400.5 | N-(2-Fluoro-5-methyl-phenyl)-2-[5-(4-isopropoxy- phenyl)-4H-[1,2,4]triazol-3-ylsulfanyl]-acetamide. It was prepared using Scheme-2 as white solid. ¹H NMR (300 MHz, DMSO-d₆) δ 1.28-1.30 (m, 6H), 2.25 (s, 3H), 4.12 (s, 2H), 4.69-4.71 (m, 1H), 6.94-7.16 (m, 4H), 7.75 (d, J = 6.0 Hz, 1H), 7.86-7.89 (m, 2H), 9.99 (s, 1H), 14.32 (s, 1H). | 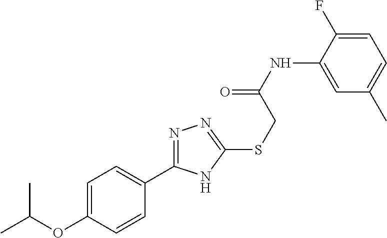 |
| ACT-CK- 131 | 389.5 | N-(3,5-Dimethyl-phenyl)-2-(5-quinolin-3-yl-4H- [1,2,4]triazol-3-ylsulfanyl)-acetamide. It was prepared using Scheme-1 as white solid. 1H NMR (300 MHz, DMSO-d₆) δ 2.22 (s, 6H), 4.17 (s, 2H), 6.70 (s, 1H), 7.23 (s, 2H), 7.69 (m, 2H), 8.06-8.10 (m, 2H), 8.88 (d, J = 1.8 Hz, 1H), 9.46 (d, J = 2.1 Hz, 1H), 10.20 (s, 1H). | 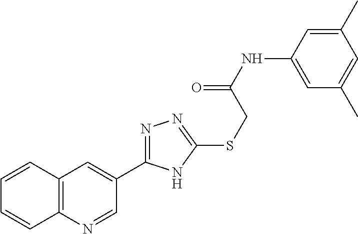 |
| ACT-CK- 132 | 434.5 | N-(2-Fluoro-5-methyl-phenyl)-2-[5-(4-phenoxy- phenyl)-4H-[1,2,4]triazol-3-ylsulfanyl]-acetamide. It was prepared using Scheme-2 as white solid. 1H NMR (300 MHz, DMSO-d₆) δ 2.24 (s, 3H), 4.13-4.22 (m, 2H), 6.93 (m, 1H), 7.09-7.24 (m, 6H), 7.42-7.47 (m, 2H), 7.74 (d, J = 6.3 Hz, 1H), 7.95-7.98 (m, 2H), 9.99 (s, 1H), 14.45 (s, 1H). | 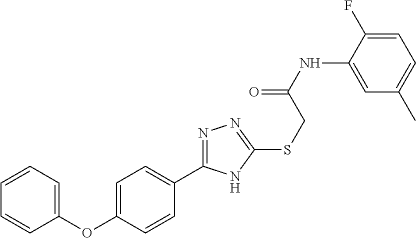 |
| ACT-CK- 133 | 430.5 | N-(3,5-Dimethyl-phenyl)-2-[5-(4-phenoxy- phenyl)-4H-[1,2,4]triazol-3-ylsulfanyl]-acetamide. It was prepared using Scheme-2 as white solid. 1H NMR (300 MHz, DMSO-d₆) δ 0.90 (t, J = 7.2 Hz, 3H), 1.57-1.65 (m, 2H), 2.22 (s, 6H), 2.58-2.63 (m, 2H), 4.07 (s, 2H), 6.70 (s, 1H), 7.20 (s, 2H), 7.31-7.34 (m, 2H), 7.85-7.88 (m, 2H), 10.14 (s, 1H), 14.33 (br s, 1H). | 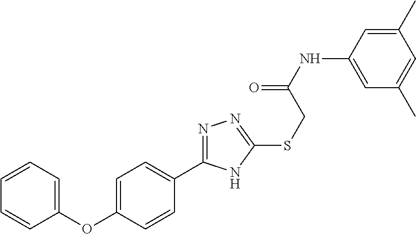 |
| ACT-CK- 135 | 411.5 | N-Benzothiazol-2-yl-2-[5-(4-ethoxy-phenyl)-4H- [1,2,4]triazol-3-ylsulfanyl]-acetamide. It was prepared using Scheme-2 as white solid. ¹H NMR (300 MHz, DMSO-d₆) δ 1.33 (t, J = 6.6 Hz, 3H), 4.06-4.23 (m, 4H), 7.00-7.03 (m, 2H), 7.31-7.45 (m, 2H), 7.79-7.97 (m, 4H), 12.67 (s, 1H), 14.32 (s, 1H). | 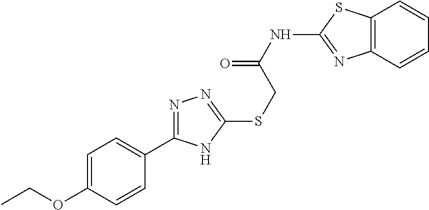 |
| ACT-CK- 137 | 389.5 | N-(3,5-Dimethyl-phenyl)-2-(5-quinolin-2-yl-4H- [1,2,4]triazol-3-ylsulfanyl)-acetamide. It was prepared using Scheme-1 as white solid. 1H NMR (300 MHz, DMSO-d₆) δ 2.22 (s, 6H), 4.14 (s, 2H), 6.70 (s, 1H), 7.23 (s, 2H), 7.67-7.72 (m, 1H), 7.84-7.89 (m, 1H), 8.06-8.20 (m, 3H), 8.56 (d, J = 7.8 Hz, 1H), 10.20 (s, 1H), 14.97 (br s, 1H). | 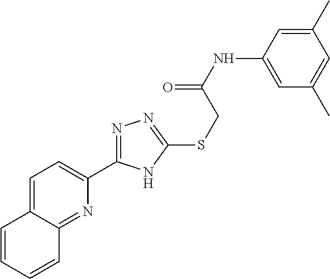 |
| ACT-CK- 138 | 382.5 | N-(3,5-Dimethyl-phenyl)-2-[5-(2-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.37 (t, J = 6.9 Hz, 3H), 2.22 (s, 6H), 4.24 (s, 2H), 4.24-4.31 (m, 2H), 6.69 (s, 1H), 7.02-7.07 (m, 1H), 7.18-7.21 (m, 3H), 7.43-7.46 (m, 1H), 7.94-7.97 (m, 1H), 10.13 (s, 1H), 13.60 (s, 1H). | 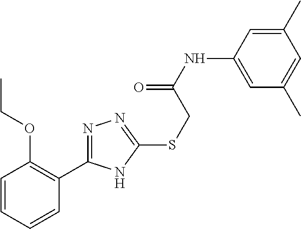 |
| ACT-CK- 139 | 444.5 | 2-[5-(4-Benzyloxy-phenyl)-4H-[1,2,4]triazol-3- ylsulfanyl]-N-(3,5-dimethyl-phenyl)-acetamide. It was prepared using Scheme-2 as white solid. 1H NMR (300 MHz, DMSO-d₆) δ 2.22 (s, 6H), 4.06 (s, 2H), 5.17 (s, 2H), 6.70 (s, 1H), 7.13-7.20 (m, 4H), 7.41-7.46 (m, 5H), 7.88-7.90 (m, 2H), 10.14 (s, 1H), 14.25 (br s, 1H). | 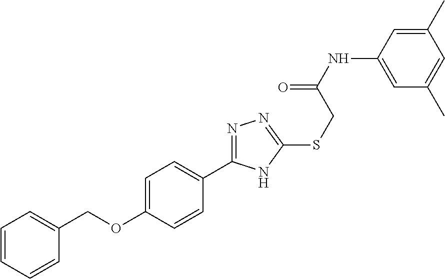 |
| ACT-CK- 140 | 368.5 | N-(3,5-Dimethyl-phenyl)-2-[5-(3-methoxy- phenyl)-4H-[1,2,4]triazol-3-ylsulfanyl]-acetamide. It was prepared using Scheme-2 as white solid. 1H NMR (300 MHz, DMSO-d₆) δ 2.21 (s, 6H), 3.79 (s, 3H), 4.08 (s, 2H), 6.69 (s, 1H), 7.03-7.06 (m, 1H), 7.21 (s, 2H), 7.41-7.55 (m, 3H), 10.15 (s, 1H), 14.43 (br s, 1H). | 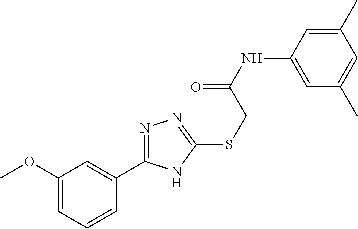 |
| ACT-CK- 141 | 382.5 | N-(3,5-Dimethyl-phenyl)-2-[5-(3-ethoxy-phenyl)- 4H-[1,2,4]triazol-3-ylsulfanyl]-acetamide. It was prepared using Scheme-2 as white solid. ¹H NMR (300 MHz, DMSO-d₆) δ 1.34-1.36 (m, 3H), 2.22 (s, 6H), 4.04-4.08 (m, 4H), 6.70 (s, 1H), 7.04 (m, 1H), 7.21 (s, 2H), 7.40-7.50 (m, 3H), 10.15 (s, 1H), 14.39 (brs, 1H). | 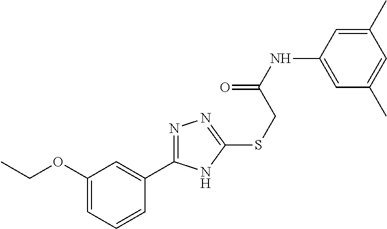 |
| ACT-CK- 142 | 396.5 | N-(3,5-Dimethyl-phenyl)-2-[5-(4-ethoxy-3-methyl- phenyl)-4H-[1,2,4]triazol-3-ylsulfanyl]-acetamide. It was prepared using Scheme-2 as brown solid, ¹H NMR (300 MHz, DMSO-d₆) δ 1.38-1.39 (m, 3H), 2.18-2.22 (m, 9H), 4.03-4.12 (m, 4H), 6.70 (s, 1H), 7.05 (d, J = 9.0 Hz, 1H), 7.21 (s, 2H), 7.74-7.75 (m, 2H), 10.13 (s, 1H), 14.23 (s, 1H). | 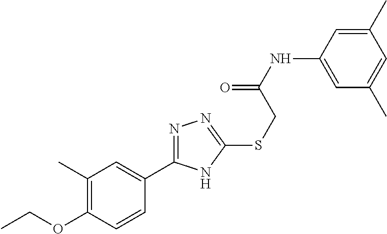 |
| ACT-CK- 143 | 383.5 | N-(3,5-Dimethyl-phenyl)-2-[5-(6-ethoxy-pyridin- 3-yl)-4H-[1,2,4]triazol-3-ylsulfanyl]-acetamide. It was prepared using Scheme-2 as brown solid. ¹H NMR (300 MHz, DMSO-d₆) δ 1.23 (t, J = 6.9 Hz, 3H), 2.21 (s, 6H), 3.96 (q, J = 6.9 Hz, 2H), 4.06 (s, 2H), 6.49-6.53 (m, 1H), 6.70 (s, 1H), 7.20 (s, 2H), 786-7.90 (m, 1H), 8.33 (s, 1H), 10.12 (s, 1H). | 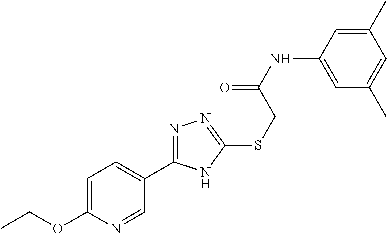 |
| ACT-CK- 146 | 354.4 | N-(3,5-Dimethyl-phenyl)-2-[5-(4-hydroxy-phenyl)- 4H-[1,2,4]triazol-3-ylsulfanyl]-acetamide. It was prepared using Scheme-1 as white solid. 1H NMR (300 MHz, DMSO-d₆) δ 2.22 (s, 6H), 4.02 (s, 2H), 6.69-6.86 (m, 3H), 7.20 (s, 2H), 7.70-7.80 (m, 2H), 10.03-10.12 (m, 2H), 14.18 (br s, 1H). | 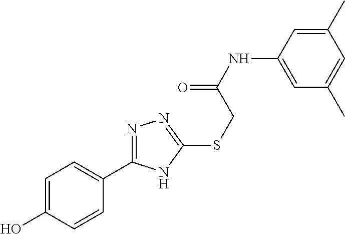 |
| ACT-CK- 147 | 383.5 | N-(4,6-Dimethyl-pyridin-2-yl)-2-[5-(4-ethoxy- phenyl)-4H-[1,2,4]triazol-3-ylsulfanyl]-acetamide.. It was prepared using Scheme-2 as white solid. ¹H NMR (300 MHz, DMSO-d₆) δ 1.34 (t, J = 6.9 Hz, 3H), 2.25 (s, 3H), 2.36 (s, 3H), 4.07-4.12 (m, 4H), 6.82 (s, 1H), 7.02-7.05 (m, 2H), 7.22 (s, 1H), 7.91-7.94 (m, 2H), 10.77 (s, 1H), 14.26 (br s, 1H). | 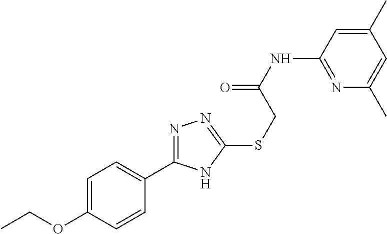 |
| ACT-CK- 148 | 384.5 | N-(4,6-Dimethyl-pyrimidin-2-yl)-2-[5-(4-ethoxy- phenyl)-4H-[1,2,4]triazol-3-ylsulfanyl]-acetamide. It was prepared using Scheme-2 as white solid. ¹H NMR (300 MHz, DMSO-d₆) δ 1.35 (t, J = 6.9 Hz, 3H), 2.36 (s, 6H), 4.06-4.10 (m, 2H), 4.25 (s, 2H), 6.95-7.08 (m, 3H), 7.88-7.91 (m, 2H), 10.72 (s, 1H), 14.28 (s, 1H). | 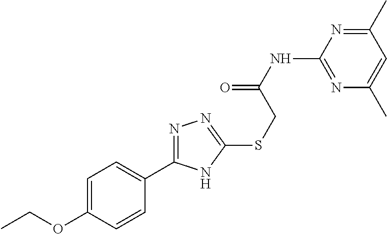 |
| ACT-CK- 149 | 426.5 | 2-[5-(3,4-Diethoxy-phenyl)-4H-[1,2,4]triazol-3- ylsulfanyl]-N-(3,5-dimethyl-phenyl)-acetamide. It was prepared using Scheme-2 as white solid. ¹H NMR (300 MHz, DMSO-d₆) δ 1.34 (t, J = 6.9 Hz, 6H), 2.22 (s, 6H), 4.01-4.09 (m, 6H), 6.70 (s, 1H), 7.00-7.10 (m, 1H), 7.20 (s, 2H), 7.49-7.50 (m, 2H), 10.14 (s, 1H), 14.23 (br s, 1H). | 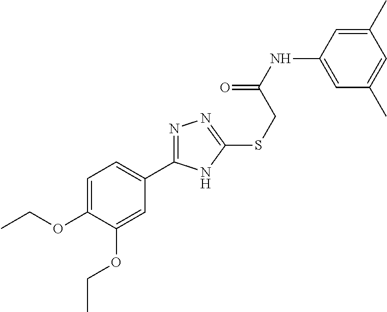 |
| ACT-CK- 150 | 396.5 | N-(3,5-Dimethyl-phenyl)-2-[5-(4-ethoxy-2-methyl- phenyl)-4H-[1,2,4]triazol-3-ylsulfanyl]-acetamide. It was prepared using Scheme-2 as white solid. only 3 mg compound was isolated. NMR could not be done. | 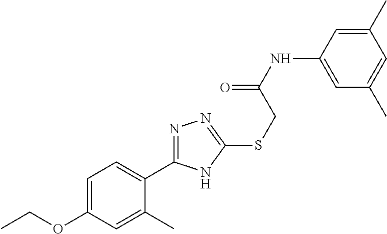 |
| ACT-CK- 151 | 383.5 | N-(2,6-Dimethyl-pyridin-4-yl)-2-[5-(4-ethoxy- phenyl)-4H-[1,2,4]triazol-3-ylsulfanyl]-acetamide. It was prepared using Scheme-2 as yellow solid; ¹H NMR (300 MHz, DMSO-d₆) δ 0.90-1.70 (m, 3H), 2.20-2.60 (m, 6H), 3.90-4.30 (m, 4H), 6.90-7.40 (m, 4H), 7.80-8.00 (m, 2H), 10.50 (s, 1H), 14.20 (br s, 1H). | 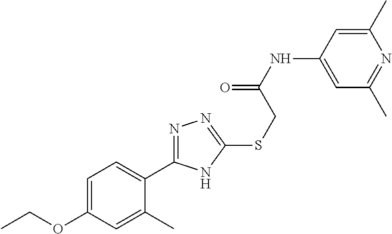 |
| ACT-CK- 152 | 490.4 | N-(3,5-Bis-trifluoromethyl-phenyl)-2-[5-(4- ethoxy-phenyl)-4H-[1,2,4]triazol-3-ylsulfanyl]- acetamide.. It was prepared using Scheme-2 as yellow solid; ¹H NMR (300 MHz, DMSO-d₆) δ 1.34 (t, J = 6.9 Hz, 3H), 4.07-4.12 (m, 4H), 7.02-7.05 (m, 2H), 7.79-7.86 (m, 3H), 8.27 (s, 2H), 10.97 (s, 1H), 14.29 (s, 1H). | 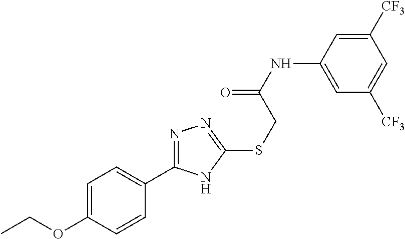 |
| ACT-CK- 153 | 396.5 | N-(3,5-Dimethyl-phenyl)-2-[5-(4-ethoxy-phenyl)- 4H-[1,2,4]triazol-3-ylsulfanyl]-N-methyl- acetamide. It was prepared using Scheme-2 as white solid; ¹H NMR (300 MHz, DMSO-d₆) δ 1.34 (t, J = 6.9 Hz, 3H), 2.27 (s, 6H), 3.16 (s, 3H), 3.89 (s, 2H), 4.05-4.10 (m, 2H), 6.99-7.06 (m, 5H), 7.79-7.82 (m, 2H), 14.16 (s, 1H). | 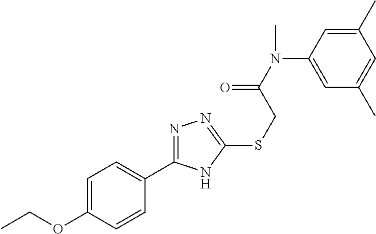 |
| ACT-CK- 154 | 400.5 | N-(3,5-Dimethyl-phenyl)-2-[5-(4-ethoxy-3-fluoro- phenyl)-4H-[1,2,4]triazol-3-ylsulfanyl]acetamide. It was prepared using Scheme-2 as white solid. ¹H NMR (300 MHz, DMSO-d₆) δ 1.37 (t, J = 6.9 Hz, 3H), 2.22 (s, 6H), 4.07-4.18 (m, 4H), 6.70 (s, 1H), 7.20-7.35 (m, 3H), 7.71-7.74 (m, 2H), 10.13 (s, 1H), 14.35 (br s, 1H). | 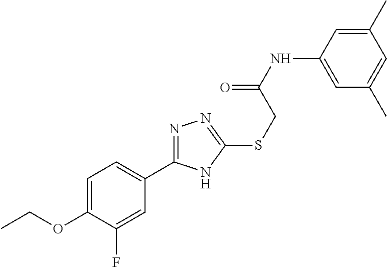 |
| ACT-CK- 155 | 398.5 | N-(3,5-Dimethyl-phenyl)-2-{5-[4-(2-hydroxy- ethoxy)-phenyl]-4H-[1,2,4]triazol-3-ylsulfanyl}- acetamide. It was prepared using Scheme-2 as white solid. 1H NMR (300 MHz, DMSO-d₆) δ 2.21 (s, 6H), 3.75 (s, 2H), 4.04-4.12 (m, 4H), 6.70-6.87 (m, 3H), 7.18 (s, 2H), 7.60-7.78 (m, 2H), 10.18 (s, 1H), 14.14 (br s, 1H). | 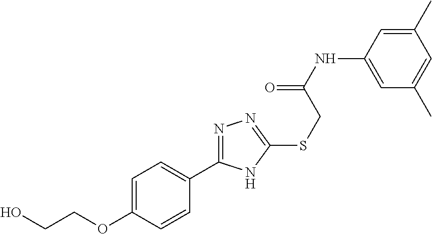 |
| ACT-CK- 156 | 368.5 | 2-[5-(4-Ethoxy-phenyl)-4H-[1,2,4]triazol-3- ylsulfanyl]-N-m-tolyl-acetamide. It was prepared using Scheme-2 as off-white solid; ¹H NMR (300 MHz, DMSO-d₆) δ 1.34 (t, J = 6.9 Hz, 3H), 2.26 (s, 3H), 4.05-4.12 (m, 4H), 6.86-6.89 (m, 1H), 7.03-7.06 (m, 2H), 7.16-7.21 (m, 1H), 7.35-7.43 (m, 2H), 7.86-7.89 (m, 2H), 10.22 (s, 1H), 14.23 (br s, 1H). | 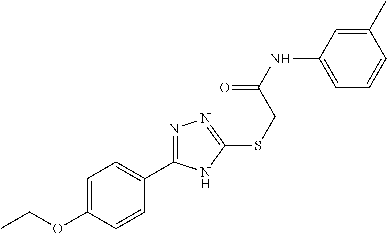 |
| ACT-CK- 157 | 384.5 | 2-[5-(4-Ethoxy-phenyl)-4H-[1,2,4]triazol-3- ylsulfanyl]-N-(3-methoxy-phenyl)-acetamide. It was prepared using Scheme-2 as white solid. ¹H NMR (300 MHz, DMSO-d₆) δ 1.34 (t, J = 6.9 Hz, 3H), 3.71 (s, 3H), 4.07-4.12 (m, 4H), 6.64 (dd, J = 8.1, 2.4 Hz, 1H), 7.03-7.10 (m, 3H), 7.18-7.29 (m, 2H), 7.86-7.88 (m, 2H), 10.29 (s, 1H), 14.24 (br s, 1H). | 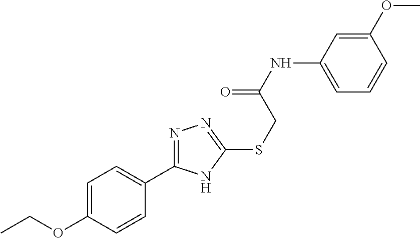 |
| ACT-CK- 158 | 398.4 | 3-{2-[5-(4-Ethoxy-phenyl)-4H-[1,2,4]triazol-3- ylsulfanyl]-acetylamino}-benzoic acid. It was prepared using Scheme-2 as white solid. ¹H NMR (300 MHz, DMSO-d₆) δ 1.34 (t, J = 6.9 Hz, 3H), 4.07-4.09 (m, 4H), 7.03-7.05 (m, 2H), 7.44-7.47 (m, 1H), 7.62-7.65 (m, 1H), 7.80-7.88 (m, 3H), 8.24 (s, 1H), 10.50 (s, 1H), 12.98 (br s, 1H), 14.29 (br s, 1H). | 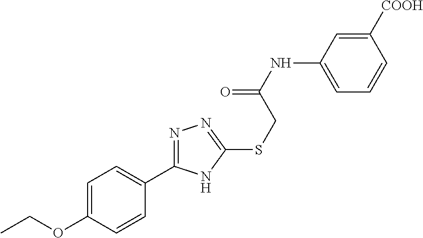 |
| ACT-CK- 159 | 404.4 | 2-[5-(4-Ethoxy-3-fluoro-phenyl)-4H-[1,2,4]triazol- 3-ylsulfanyl]-N-(3-fluoro-5-methyl-phenyl)- acetamide. It was prepared using Scheme-2 as white solid. ¹H NMR (300 MHz, DMSO-d₆) δ 1.37 (t, J= 6.9 Hz, 3H), 2.28 (s, 3H), 4.09-4.20 (m, 4H), 6.73 (d, J = 9.6 Hz, 1H), 7.14 (s, 1H), 7.26-7.37 (m, 2H), 7.71-7.74 (m, 2H), 10.43 (s, 1H), 14.32 (br s, 1H). | 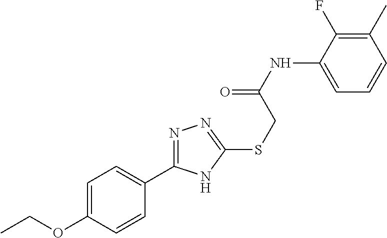 |
| ACT-CK- 160 | 386.4 | 2-[5-(4-Ethoxy-phenyl)-4H-[1,2,4]triazol-3- ylsulfanyl]-N-(2-fluoro-3-methyl-phenyl)- acetamide. It was prepared using Scheme-2 as white solid. ¹H NMR (300 MHz, DMSO-d₆) δ 1.44 (t, J = 6.9 Hz, 3H), 2.18 (s, 3H), 3.96 (s, 2H), 4.08 (q, J = 6.9 Hz, 2H), 6.84-7.00 (m, 4H), 7.87-7.90 (m, 2H), 8.09 (t, J = 7.2 Hz, 1H), 9.71 (s, 1H), 11.80 (br s, 1H). | 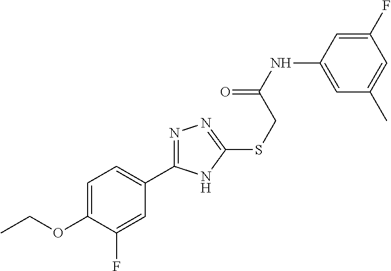 |
| ACT-CK- 161 | 404.4 | 2-[5-(4-Ethoxy-3-fluoro-phenyl)-4H-[1,2,4]triazol- 3-ylsulfanyl]-N-(2-fluoro-3-methyl-phenyl)- acetamide. It was prepared using Scheme-2 as white solid. ¹H NMR (300 MHz, DMSO-d₆) δ 1.37 (t, J = 6.9 Hz, 3H), 2.23 (s, 3H), 4.13-4.20 (m, 4H), 6.99-7.06 (m, 2H), 7.29 (t, J = 8.7 Hz, 1H), 7.72-7.76 (m, 3H), 10.01 (s, 1H), 14.30 (br s, 1H). | 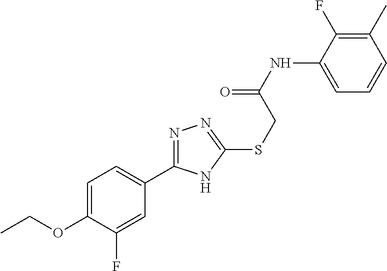 |
| ACT-CK- 162 | 386.4 | 2-[5-(4-Ethoxy-phenyl)-4H-[1,2,4]triazol-3- ylsulfanyl]-N-(3-fluoro-5-methyl-phenyl)- acetamide. It was prepared using Scheme-2 as white solid. ¹H NMR (300 MHz, DMSO-d₆) δ 1.34 (t, J = 6.9 Hz, 3H), 2.28 (s, 3H), 4.05-4.12 (m, 4H), 6.73 (d, J = 9.6 Hz, 1H), 7.03 (s, 1H), 7.06 (s, 1H), 7.14 (s, 1H), 7.35 (m, 1H), 7.85-7.88 (m, 2H), 10.43 (s, 1H), 14.22 (br s, 1H). | 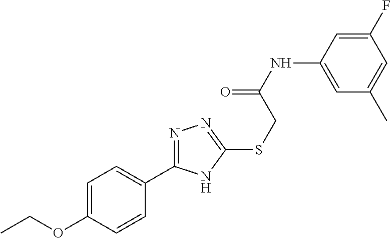 |
| ACT-CK- 163 | 442.4 | 5-{2-[5-(4-Ethoxy-phenyl)-4H-[1,2,4]triazol-3- ylsulfanyl]-acetylamino}-isophthalic acid. It was prepared using Scheme-2 as light brown solid; ¹H NMR (300 MHz, DMSO-d₆) δ 1.33 (t, J = 6.6 Hz, 3H), 4.00-4.11 (m, 5H), 6.92-7.03 (m, 2H), 7.89-7.92 (m, 2H), 8.22 (s, 3H), 10.61 (br s, 1H). | 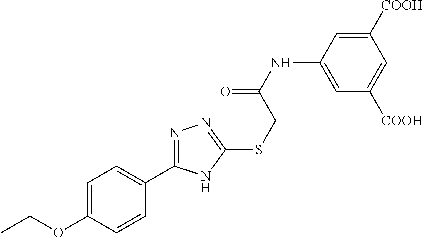 |
| ACT-CK- 164 | 416.5 | 2-[5-(4-Ethoxy-3-fluoro-phenyl)-4H-[1,2,4]triazol- 3-ylsulfanyl]-N-(2-hydroxy-3,5-dimethyl-phenyl)- acetamide. It was prepared using Scheme-2 as white solid. ¹H NMR (300 MHz, DMSO-d₆) δ 1.37 (t, J = 6.6 Hz, 3H), 2.12-2.14 (m, 6H), 4.12-4.18 (m, 4H), 6.71 (s, 1H), 7.23-7.29 (m, 2H), 7.73-7.76 (m, 2H), 8.63 (br s, 1H), 9.80 (s, 1H), 14.34 (br s, 1H). | 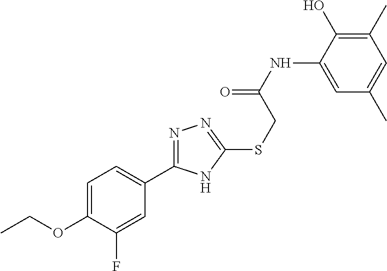 |
| ACT-CK- 165 | 398.5 | N-(3,5-Dimethyl-phenyl)-2-[5-(4-ethylsulfanyl- phenyl)-4H-[1,2,4]triazol-3-ylsulfanyl]-acetamide. It was prepared using Scheme-1 as white solid ¹H NMR (300 MHz, DMSO-d₆) δ 1.27 (t, J = 6.0 Hz, 3H), 2.22 (s, 6H), 3.04-3.06 (m, 2H), 4.08 (s, 2H), 6.70 (s, 1H), 7.20 (s, 2H), 7.40 (d, J = 9.0 Hz, 2H), 7.87 (d, J = 9.0 Hz, 2H), 10.14 (s, 1H), 14.23 (br s, 1H). | 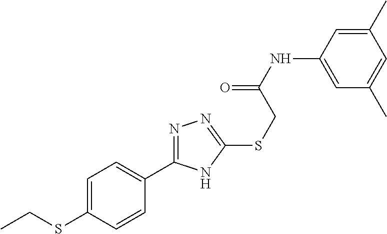 |
| ACT-CK- 166 | 353.4 | 2-[5-(4-Amino-phenyl)-4H-[1,2,4]triazol-3- ylsulfanyl]-N-(3,5-dimethyl-phenyl)-acetamide. It was prepared using Scheme-1 as off-white solid; 1H NMR (300 MHz, DMSO-d₆) δ 2.22 (s, 6H), 4.07 (s, 2H), 5.63 (br s, 2H), 6.60-6.70 (m, 3H), 7.20 (s, 2H), 7.60-7.62 (m, 2H), 10.13 (s, 1H), 13.93 (br s, 1H). | 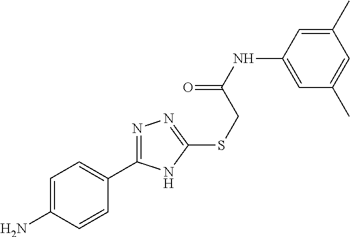 |
| ACT-CK- 167 | 383.4 | N-(3,5-Dimethyl-phenyl)-2-[5-(4-nitro-phenyl)- 4H-[1,2,4]triazol-3-ylsulfanyl]-acetamide. It was prepared using Scheme-1 as light yellow solid; 1H NMR (300 MHz, DMSO-d₆) δ 2.22 (s, 6H), 4.20 (s, 2H), 6.70 (s, 1H), 7.20 (s, 2H), 8.20-8.22 (m, 2H), 8.31-8.39 (m, 2H), 10.19 (s, 1H), 14.49 (br s, 1H). | 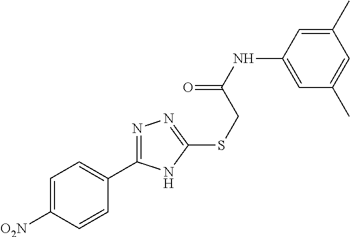 |
| ACT-CK- 169 | 488.5 | 5-{2-[5-(4-Ethoxy-3-fluoro-phenyl)-4H- [1,2,4]triazol-3-ylsulfanyl]-acetylamino}- isophthalic acid dimethyl ester. It was prepared using Scheme-2 as white solid ¹H NMR (300 MHz, DMSO-d₆) δ 1.36 (t, J = 6.9 Hz, 3H), 3.88 (s, 6H), 4.10-4.17 (m, 4H), 7.29 (br s, 1H), 7.77 (d, J = 8.1 Hz, 2H), 8.17 (s, 1H), 8.46 (d, J = 9.3 Hz, 2H), 10.75 (s, 1H), 14.42 (br s, 1H). | 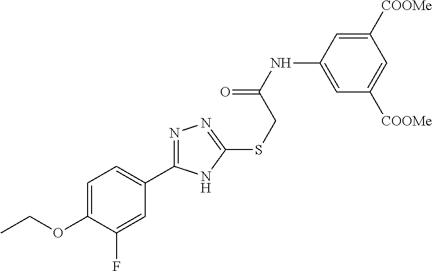 |
| ACT-CK- 170 | 417.6 | 2-[5-(4-Ethoxy-phenyl)-4H-[1,2,4]triazol-3- ylsulfanyl]-N-(2,2,6,6-tetramethyl-piperidin-4-yl)- acetamide. It was prepared using Scheme-2 as white solid. ¹H NMR (300 MHz, DMSO-d₆) δ 0.91-1.00 (m, 8H), 1.11 (s, 6H), 1.34 (t, J = 6.9 Hz, 3H), 1.58-1.64 (m, 2H), 3.80 (s, 2H), 4.09 (q, J = 6.9 Hz, 2H), 7.03 (d, J = 9 Hz, 2H), 7.87 (d, J = 9 Hz, 2H), 8.09 (d, J = 9 Hz, 1H). | 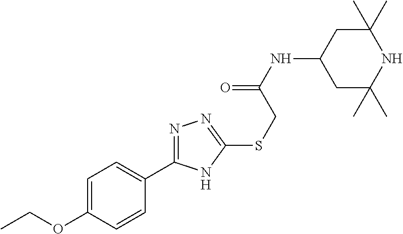 |
| ACT-CK- 171 | 435.6 | 2-[5-(4-Ethoxy-3-fluoro-phenyl)-4H-[1,2,4]triazol- 3-ylsulfanyl]-N-(2,2,6,6-tetramethyl-piperidin-4- yl)-acetamide. It was prepared using Scheme-2 as white solid ¹H NMR (300 MHz, DMSO-d₆) δ 0.97-1.14 (m, 14H), 1.36 (t, J = 8.4 Hz, 3H), 1.64 (d,, J = 8.4 Hz, 2H), 3.80 (s, 2H), 4.13-4.19 (m, 2H), 7.27 (t, J = 9 Hz, 1H), 7.72 (d, J = 9 Hz, 2H), 8.16 (d, J = 6 Hz, 1H). | 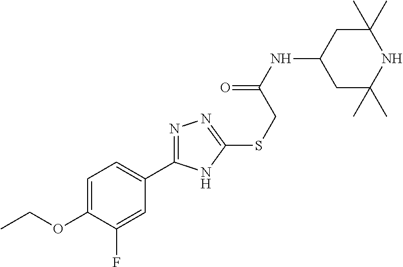 |

### II. Chemical Synthesis

The compounds of the present invention can be prepared using the method described below in Scheme 1, together with synthetic methods known by one in the art of organic synthesis and variations thereon.

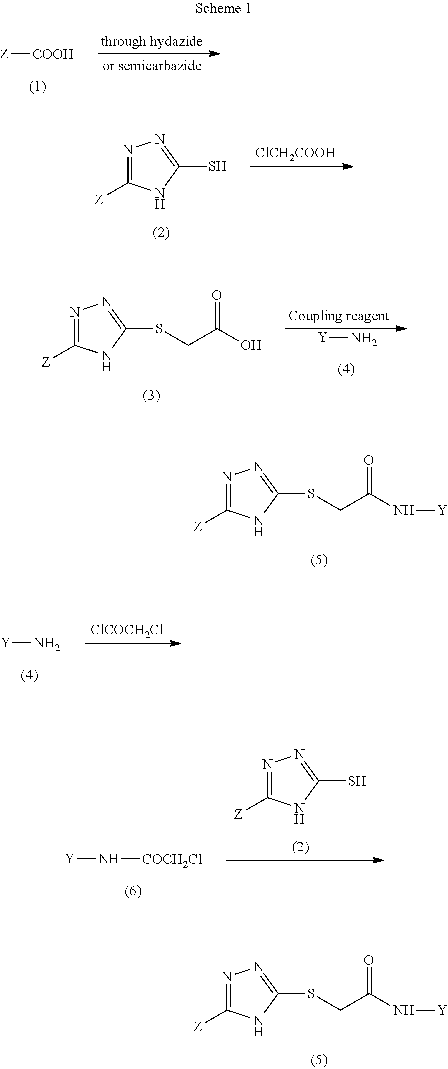

The target molecules are synthesized from the corresponding carboxylic acids (1), which are converted to triazoles (2) through hydrazide and thiourea or thiosemicarbazide reactions. Triazoles formed are converted to acetic acid derivatives (3) through chloroacetic acid, which in turn can be coupled to corresponding amines (4) to give the desired target molecules (5).

These targets also are prepared by the reaction of chloroacetylamides (5) with triazoles (2).

The precursor carboxylic acids (1) and amines (4) are either commercially available or are prepared according to the literature procedures.

More specifically, ethoxy derivatives of the present invention can be prepared according to the following Scheme 2.

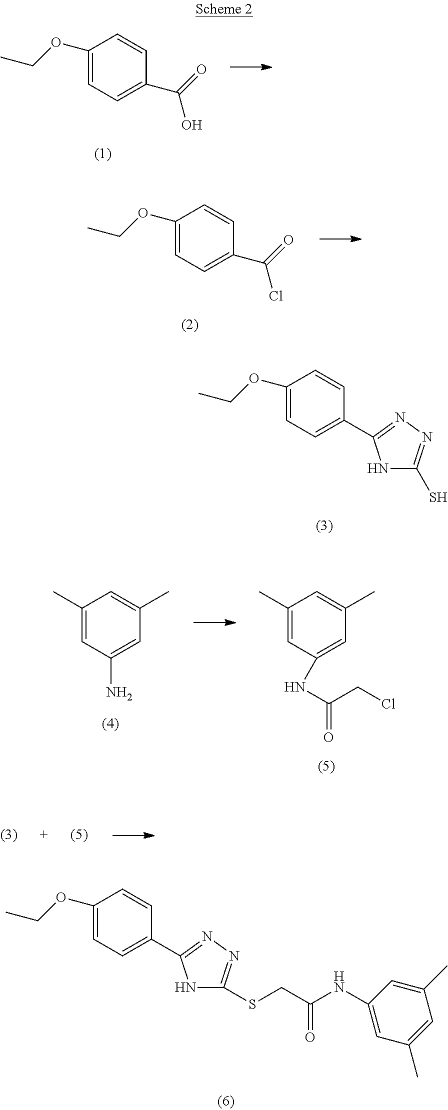

### Preparation of (2)

Oxalyl chloride (4.5 g, 36 mmole) followed by two drops DMF were added to a stirred solution of 4-ethoxybenzoic acid 1 (3.0 g, 18 mmole) in dichloromethane (46 ml). Solution was stirred for 3 h at 25° C. Completion of the reaction was checked by TLC (CHCl₃:MeOH:9:1). Reaction mixture was then concentrated under vacuum to obtain compound 2.

### Preparation of (3)

Compound 2 (3.12 g 16.89 mmole) was added dropwise to a stirred solution of thiosemicarbazide (1.54 g, 16.89 mmole) in 2N NaOH (20 mL) at 0-5° C. Reaction mixture was then stirred at room temperature for 2 h and heated at reflux for 3 h. Reaction mixture was cooled to room temperature and 2 mL of 10N NaOH was added to the reaction mixture. Resulting solution was filtered and acidified with concentrated HCl. The precipitate obtained was collected by filtration, which was recrystallized from ethanol to yield the desired compound 3 as white solid, yield 55%.

### Preparation of (5):

Chloroacetyl chloride (54.38 mmole) was added drop wise to a stirred solution of 3,5-dimethyl aniline 5 (54.38 mmole) in acetonitrile (70 mL) at ice bath temperature. Reaction mixture was then heated at reflux until the gas (HCl) ceased to evolve. The reaction mixture was then cooled to ambient temperature and the solvent was removed in vacuo. The solid residue was washed with acetonitrile (3×8 mL), filtered and dried under vacuum to give compound 5 as a white powder. Yield 79%

### Preparation of (6):

Compound 5 (0.2 g, 1.01 mmole) was added to a solution of compound 3 (0.224 g, 1.01 mmole) and NaOH (0.044 g, 1.11 mmole) in methanol (10 mL). The reaction mixture was stirred overnight at room temperature, solvent was removed and the crude was purified by column chromatography over silica gel using ethyl acetate-hexane solvent system. Compound 6 was obtained as white solid, Yield 60%. MH⁺—383.3, ¹H NMR (DMSO-D₆): 14.27 (brs, 1H), 10.12 (brs, 1H), 7.90-7.84 (m, 2H), 7.19 (s, 2H), 7.08-6.99 (m, 2H), 6.68 (s, 1H), 4.14-4.01 (m, 4H), 2.22 (s, 6H), 1.34 (t, 3H), HPLC −100%.

In another embodiment, compounds of the invention having NH—C₂H₅ in place of O—C₂H₅ can be prepared according to the following Scheme 3.

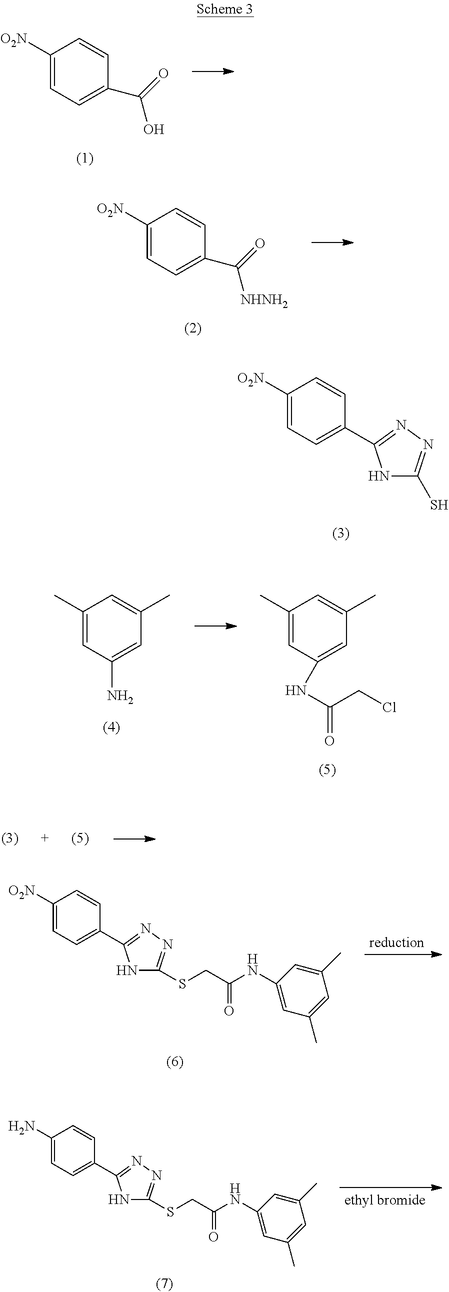

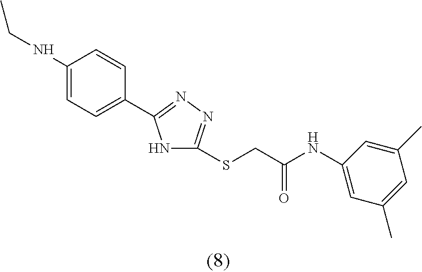

### III. Pharmaceutical Compositions

The compounds of Formula I, the pharmaceutically acceptable salts thereof, the prodrugs corresponding to compounds of Formula I, and the pharmaceutically acceptable salts thereof, are all referred to herein as “anti-cancer compounds.” The Formula I compounds disclosed herein can be administered to a subject either alone, or as part of a pharmaceutical composition.

Pharmaceutical compositions comprising the aforementioned anti-cancer compounds also are provided herein. These pharmaceutical compositions comprise active compounds as described herein, in a pharmaceutically acceptable carrier. Pharmaceutical formulations can be prepared for oral, intravenous, parenteral, or aerosol administration as discussed in greater detail below. Also, the compounds of the present invention provide such anti-cancer compounds that can be reconstituted to form pharmaceutically acceptable compositions (including compositions pharmaceutically acceptable in humans) for administration.

The term “carrier,” as used herein, includes pharmaceutically acceptable carriers, excipients, or stabilizers which are nontoxic to the cell or mammal being exposed thereto at the dosages and concentrations employed.

The therapeutically effective dosage of any specific anti-cancer compound, the use of which is within the scope of embodiments described herein, will vary somewhat from compound to compound, and subject to subject, and will depend upon the condition of the subject and the route of delivery. As a general proposition, a dosage from about 0.1 to about 500 mg/kg will have therapeutic efficacy, with all weights being calculated based upon the weight of the active compound, including the cases where a salt is employed.

In accordance with the presently disclosed methods, pharmaceutically active compounds as described herein can be administered orally as a solid, liquid, or gel, or can be administered intramuscularly or intravenously as a solution, suspension, or emulsion. Alternatively, the compounds or salts also can be administered by inhalation, intravenously, or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical compositions suitable for intravenous or intramuscular injection are further embodiments provided herein. The pharmaceutical compositions comprise a compound of Formula I described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is preferably done by an aseptic method. Sterilized closures can then be placed on the vials and, if desired, the vial contents can be lyophilized.

In addition to compounds of Formula I, or their salts or prodrugs, the pharmaceutical compositions can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical compositions described herein can be lyophilized using techniques well known in the art.

In yet another embodiment of the subject matter described herein, there is provided an injectable, stable, sterile formulation comprising a compound of Formula I, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the compound or salt is substantially water-insoluble, a sufficient amount of emulsifying agent, which is physiologically acceptable, can be employed in sufficient quantity to emulsify the compound or salt in an aqueous carrier.

Other pharmaceutical compositions can be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof.

Additional embodiments provided herein include liposomal formulations of the active compounds disclosed herein. The technology for forming liposomal suspensions is well known in the art.

Pharmaceutical compositions also are provided which are suitable for administration as an aerosol by inhalation. These compositions comprise a solution or suspension of a desired compound described herein or a salt thereof, or a plurality of solid particles of the compound or salt. The desired composition can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns.

As indicated, both water-soluble and water-insoluble active compounds are provided. As used herein, the term “water-soluble” is meant to define any composition that is soluble in water in an amount of about 10 mg/mL, or greater. Also, as used herein, the term “water-insoluble” is meant to define any composition that has solubility in water of less than about 1 mg/mL. In some embodiments, water-soluble compounds or salts can be desirable whereas in other embodiments water-insoluble compounds or salts likewise can be desirable.

### IV. Methods of Inhibiting Cell Proliferation and Treating Cancer

The compounds of the present invention and compositions including them are useful for inhibiting cell proliferation.

In some embodiments, the methods for inhibiting cell proliferation or treating a cancer comprise administering to a subject in need thereof an anti-cancer compound as described herein. These active compounds, as set forth above, include the compounds of Formula I, their corresponding prodrugs, and pharmaceutically acceptable salts of the compounds and prodrugs. In some embodiments, the active compound is present in a pharmaceutical formulation as described hereinabove.

The presently disclosed compounds can provide therapy for a wide variety of tumors and cancers including skin cancers, connective tissue cancers, adipose cancers, breast cancers, lung cancers, stomach cancers, pancreatic cancers, ovarian cancers, cervical cancers, uterine cancers, anogenital cancers, kidney cancers, bladder cancers, colon cancers, prostate cancers, central nervous system (CNS) cancers, retinal cancer, blood, and lymphoid cancers.

An “effective amount” is defined herein in relation to the treatment of cancers is an amount that will decrease, reduce, inhibit, or otherwise abrogate the growth of a cancer cell or tumor. In some embodiments, the compound of Formula I can be delivered regionally to a particular affected region or regions of the subject's body. In some embodiments, wherein such treatment is considered more suitable, the compound of Formula I can be administered systemically. For example, the compound can be administered orally or intravenously.

In addition, it will be appreciated that therapeutic benefits for the treatment of cancer can be realized by combining treatment with a compound or compounds of the compounds of the present invention with one or more additional anti-cancer agents or treatments. The choice of such combinations will depend on various factors including, but not limited to, the type of disease, the age and general health of the subject, the aggressiveness of disease progression, and the ability of the subject to tolerate the agents that comprise the combination.

Thus, a variety of chemical compounds, also described as “anti-neoplastic” agents or “chemotherapeutic agents” can be used in combination with one or more of the novel anti-cancer compounds of the presently described subject matter. Such compounds include, but are not limited to, alkylating agents, DNA intercalators, protein synthesis inhibitors, inhibitors of DNA or RNA synthesis, DNA base analogs, topoisomerase inhibitors, anti-angiogenesis agents, and telomerase inhibitors or telomeric DNA binding compounds. For example, suitable alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride; melphalan, novembichine, phenesterine, prednimustine, trofosfamide, and uracil mustard; nitroso ureas, such as carmustine, chlorozotocin, fotemustine, lomustine, nimustine, and ranimustine.

Antibiotics used in the treatment of cancer include dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, Shiga toxin, showdomycin, sparsomycin, spectinomycin, streptomycin, tetracycline, thiostrepton, and trimethoprim. Inhibitors of DNA synthesis, including alkylating agents such as dimethyl sulfate, mitomycin C, nitrogen and sulfur mustards, intercalating agents, such as acridine dyes, actinomycins, adriamycin, anthracenes, benzopyrene, ethidium bromide, propidium diiodide-intertwining, and agents, such as distamycin and netropsin, also can be combined with compounds of the compounds of the present invention in pharmaceutical compositions. Topoisomerase inhibitors, such as coumermycin, nalidixic acid, novobiocin, and oxolinic acid, inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be combined with the inhibitor compounds of the compounds of the present invention to provide a suitable cancer treatment.

Thus, current chemotherapeutic agents that can be used in a combination treatment with the choline kinase inhibitor compounds of the present invention include, but are not limited to, adrimycin, 5-fluorouracil (5FU), etoposide, camptothecin, actinomycin-D, mitomycin, cisplatin, hydrogen peroxide, carboplatin, procarbazine, mechlorethamine, cyclophosphamide, ifosfamide, melphalan, chjlorambucil, bisulfan, nitrosurea, dactinomycin, duanorubicin, doxorubicin, bleomycin, plicomycin, tamoxifen, taxol, transplatimun, vinblastin, and methotrexate, and the like.

Combination treatments involving compounds of the compounds of the present invention can be tested and another therapeutic agent, such as another chemotherapeutic agent can be achieved by using both agents at the same time. Alternatively, treatment with compounds of the compounds of the present invention can precede or follow treatment with the other agent by intervals ranging from minutes to weeks.

The compounds of the compounds of the present invention can be tested to measure their ability to inhibit growth of cancer cells, to induce apoptosis of the cancer cells, to reduce tumor burden, and to inhibit metastases. For example, one can measure cell growth according to the MTT assay, the Alamar Blue assay or the cell Titer Glow assay. Growth assays as measured by the methods listed above are well known in the art and measure either directly the number of viable cells (MTT and Alamar Blue assays) or the intracellular levels of ATP, inferring from the viability of cells (Cell Titer glow assay).

In one embodiment, a compound or its enantiomeric or diastereomeric form or a pharmaceutically acceptable salt, prodrug, or metabolite thereof is provided, said compound having the formula:

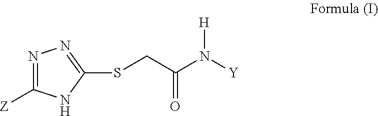

wherein:

Z is selected from the group consisting of:

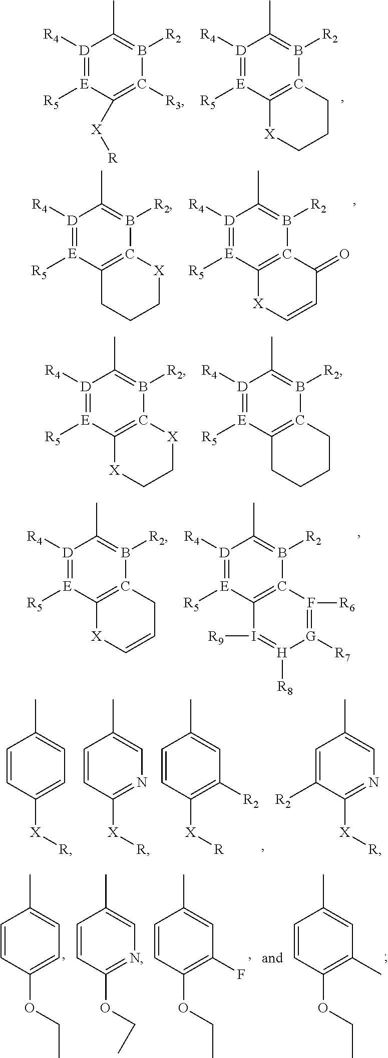

Y is selected from the group consisting of:

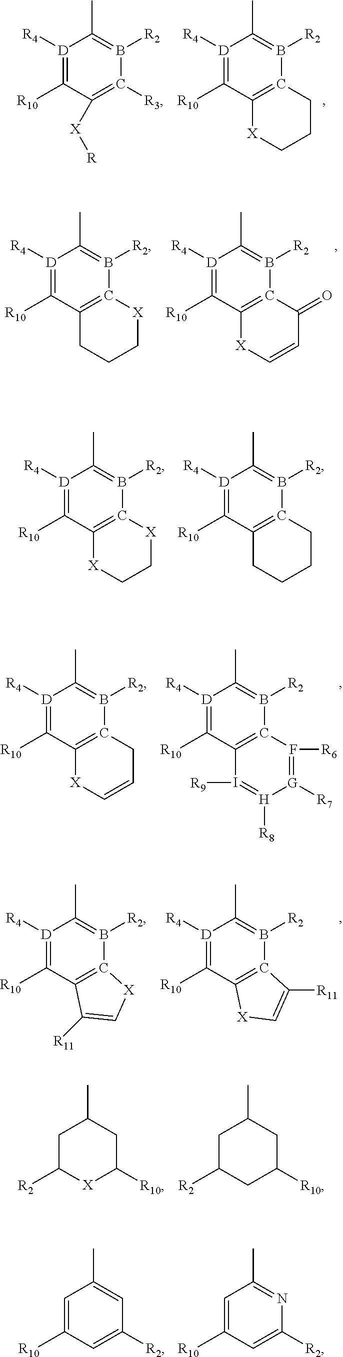

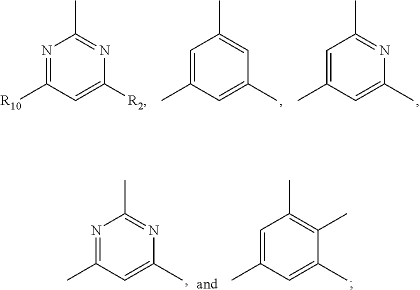

X is O, S, NH, or N-alkyl;

R is alkyl or substituted alkyl;

B, C, D, E, F, G, H, and I are independently selected from the group consisting of N or C substituted with one of R₂, R₃, R₄, R₅, R₆, R₇, R₈, or R₉, wherein, if B, C, D, E, F, G, H, or I are N, then R₂, R₃, R₄, R₅, R₆, R₇, R₈, or R₉ represent the free electron pair at the N atom;

R₂, R₃, R₄, R₅, R₆, R₇, R₈, or R₉ are independently, when attached to an N atom, a free electron pair, or, when attached to C, selected from the group consisting of hydrogen, halogen, cyano, nitro, straight-chain or branched (C₁-C₆)-alkyl, straight-chain or branched (C₁-C₆)-alkyl substituted with one or more halogen atoms, straight-chain or branched (C₁-C₆)-alkoxy substituted with one or more halogen atoms, (C₂-C₆)-alkenyl, (C₂-C₆)-alkynyl, (C₃-C₈)-cycloalkyl, straight-chain or branched (C₁-C₆)-alkoxy, straight-chain or branched (C₁-C₆)-alkylenedioxy, (C₁-C₆)-alkoxycarbonyl, (C₁-C₆)-alkoxycarbonyloxy, (C₁-C₆)-alkylcarbonyl, (C₁-C₆)-alkylcarbonyloxy, (C₁-C₆)-alkylthio, (C₁-C₆)-alkylsulfinyl, (C₁-C₆)-alkylsulfonyl, carboxyl, (C₁-C₆)-alkyl carboxylate, carboxamide, N—(C₁-C₄)-alkyl-carboxamide, N,N-di-(C₁-C₄)-alkyl-carboxamide, (C₁-C₆)-alkoxy-(C₁-C₆)-alkyl, amino, mono-(C₁-C₆)-alkylamino, N,N-di-(C₁-C₆)-alkylamino, where two C₁-C₆-alkyl radicals together may form a ring, which optionally contains one or more of NH, N—(C₁-C₆)-alkyl, O or S, (C₆-C₁₄)-aryl, (C₆-C₁₄)-aryloxy, (C₆-C₁₄)-aryl-(C₁-C₄)-alkyl, (C₆-C₁₄)-aryl-(C₁-C₄)-alkoxy-(C₁-C₄)-alkyl, (C₁-C₆)-alkylcarbonyl, (C₁-C₆)-alkoxycarbonyl, hydroxyl, wherein two directly adjacent radicals may be attached to one another; and

R₁₀ is alkyl or substituted alkyl.

With respect to Z, in a more specific embodiment, Z is selected from the group consisting of

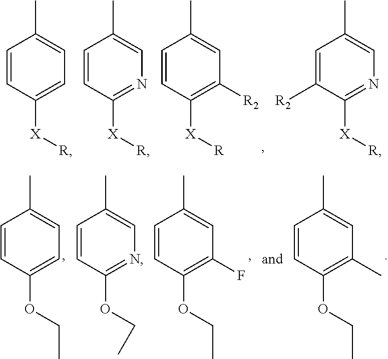

In a more specific embodiment, Z is selected from the group consisting of

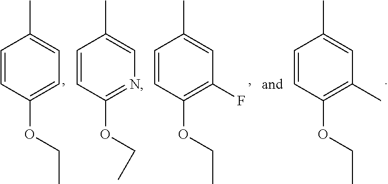

With respect to Y, in another specific embodiment, Y is selected from the group consisting of

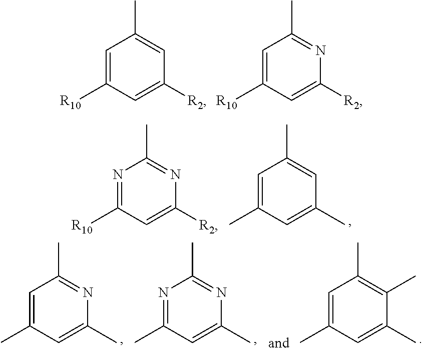

In a more specific embodiment, Y is selected from the group consisting of

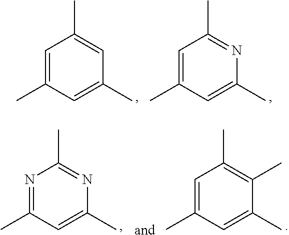

With respect to R₂-R₉, in a specific embodiment, the straight-chain or branched (C₁-C₆)-alkyl substituted with one or more halogen atoms is trifluoromethyl. In another specific embodiment, the straight-chain or branched (C₁-C₆)-alkoxy substituted with one or more halogen atoms is trifluoromethoxy. In still another specific embodiment, the straight-chain or branched (C₁-C₆)-alkoxy is methoxy. In another specific embodiment, the straight-chain or branched (C₁-C₆)-alkylenedioxy is methylenedioxy.

In a very specific embodiment, the compound is selected from the group consisting of:
N-(3,5-Dimethyl-phenyl)-2-[5-(4-ethoxy-phenyl)-4H-[1,2,4]triazol-3-ylsulfanyl]-acetamide (ACT-CK-050); 2-[5-(4-Methoxy-phenyl)-4H-[1,2,4]triazol-3-ylsulfanyl]-N-(5-methyl-thiazol-2-yl)-acetamide (ACT-CK-099); N-(3,5-Dimethyl-phenyl)-2-[5-(1H-indol-2-yl)-4H-[1,2,4]triazol-3-ylsulfanyl]-acetamide (ACT-CK-124); N-(3,5-Dimethyl-phenyl)-2-[5-(4-ethoxy-3-methyl-phenyl)-4H-[1,2,4]triazol-3-ylsulfanyl]-acetamide (ACT-CK-142); N-(3,5-Dimethyl-phenyl)-2-[5-(6-ethoxy-pyridin-3-yl)-4H-[1,2,4]triazol-3-ylsulfanyl]-acetamide (ACT-CK-143);
N-(4,6-Dimethyl-pyridin-2-yl)-2-[5-(4-ethoxy-phenyl)-4H-[1,2,4]triazol-3-ylsulfanyl]-acetamide (ACT-CK-147); N-(4,6-Dimethyl-pyrimidin-2-yl)-2-[5-(4-ethoxy-phenyl)-4H-[1,2,4]triazol-3-ylsulfanyl]-acetamide (ACT-CK-148); N-(3,5-Dimethyl-phenyl)-2-[5-(4-ethoxy-3-fluoro-phenyl)-4H-[1,2,4]triazol-3-ylsulfanyl]-acetamide (ACT-CK-154); 2-[5-(4-Ethoxy-3-fluoro-phenyl)-4H-[1,2,4]triazol-3-ylsulfanyl]-N-(2-hydroxy-3,5-dimethyl-phenyl)-acetamide (ACT-CK-164); and N-(3,5-Dimethyl-phenyl)-2-[5-(4-ethylsulfanyl-phenyl)-4H-[1,2,4]triazol-3-ylsulfanyl]-acetamide (ACT-CK-165).

In another embodiment, a pharmaceutical composition for the treatment of cancer is provided, the composition comprising a Formula I compound and at least one pharmaceutically acceptable carrier. In another embodiment, the pharmaceutical composition further comprises one or more additional chemotherapeutic agents.

In another embodiment, a method of treating cancer is provided, the method comprising administering to a subject in need thereof an effective amount of a Formula I compound.

In another embodiment, a method of inhibiting enzymatic activity of choline kinase in a cell is provided, the method comprising administering an effective amount of a Formula I compound.

In still another embodiment, a method of treating a tumor is provided, the method comprising administering to a subject in need thereof an effective amount of one or more Formula I compounds.

### EXAMPLES

The following examples are given by way of illustration only and are in no way intended to limit on the scope of the present invention.

### Example 1

Inhibition of Cancer Cell Proliferation

The ability of the compounds described herein to kill or inhibit the proliferation of cancer cells was measured using the Alamar Blue assay using a 72 hours exposure. Results for different cancer cell lines are shown in, the Table 2 below (hematopoietic cell lines U937 and K562; MDA-MB-231 a breast cancer cell line; and NCI-H82 and Calu-6, two lung cancer cell lines):

TABLE 2
A: Inhibition of cell proliferation

| | IC50s | | | | | |
|---|---|---|---|---|---|---|
| | | MDA- | | NCI- | | |
| Compounds | K562 | MB231 | U937 | H82 | Calu-6 | — |
| ACT-CK-050 | 0.04 | 18.5 | 0.06 | 0.09 | 0.06 | |
| ACT-CK-061 | 28.1 | >100 | | | | |
| ACT-CK-063 | 37 | 78.7 | | | | |
| ACT-CK-064 | 39.5 | 70.6 | | | | |
| ACT-CK-066 | 11.4 | 76.2 | | | | |
| ACT-CK-067 | 1.6 | 19.2 | | | | |
| ACT-CK-070 | 32.1 | 68.2 | | | | |
| ACT-CK-071 | 0.81 | 40.2 | | | | |
| ACT-CK-076 | 14.64 | 21.25 | | | | |
| ACT-CK-085 | 4.4 | 16.28 | | | | |
| ACT-CK-087 | 8.86 | 10.6 | | | | |
| ACT-CK-093 | 6.16 | 15.71 | | | | |
| ACT-CK-096 | 34 | 149 | | | | |
| ACT-CK-097 | 3.78 | 12 | | | | |
| ACT-CK-118 | 1.4 | 5.6 | | | | |
| ACT-CK-119 | 28 | 11 | | | | |
| ACT-CK-120 | 43 | 9 | | | | |
| ACT-CK-121 | 39 | 10 | | | | |
| ACT-CK-124 | 23 | 16 | | | | |
| ACT-CK-136 | 10 | | 3.2 | 10 | 1.4 | |
| ACT-CK-140 | 41 | 79 | 20.4 | | 41.6 | |
| ACT-CK-141 | 39 | 88 | 49 | | 43 | |
| ACT-CK-142 | 0.12 | 50 | 0.035 | | 0.09 | |
| ACT-CK-144 | 34 | 47 | 46 | | 37.8 | |
| ACT-CK-145 | 1.2 | 16.8 | 0.56 | | 0.64 | |
| ACT-CK-146 | 31.7 | 91 | 34.1 | | 54 | |
| ACT-CK-147 | 0.97 | 53 | 0.63 | | 0.74 | |
| ACT-CK-148 | 0.72 | 29 | 0.5 | 8.1 | 0.47 | |
| ACT-CK-154 | 0.8 | 2.6 | 0.02 | 0.16 | 0.03 | |
| ACT-CK-156 | 3.27 | 7.0 | 1.1 | 1.7 | 1.7 | |

B: Inhibition of cell proliferation results
for ACT-CK-050 in a panel of cell lines

| | |
|---|---|
| IC₅₀ μM | ACT-CK-050 |

| | | |
|---|---|---|
| Cell lines | Aver | Stdev |

| | | | |
|---|---|---|---|
| K562 | Leukemia | 0.043 | 0.006 |
| MDA-MB-231 | Breast | 18.53 | 1.75 |
| Jurkat | Leukemia | 10.40 | 1.39 |
| H209 | Lung | 0.367 | 0.045 |
| Hela | Cervical | 0.022 | 0.009 |
| HT-29 | Colon | 89.67 | 11.01 |
| HCT-116 | Colon | 0.523 | 0.040 |
| U937 | lymphoma | 0.063 | 0.012 |
| NCI-H82 | Lung | 0.096 | 0.001 |
| NCI-H2122 | Lung | 0.173 | 0.021 |
| Colo-205 | Colorectal | 0.240 | 0.010 |
| Calu-6 | Lung | 0.057 | 0.006 |
| Hep G2 | Liver | 0.163 | 0.015 |
| A431 | epidermi | 0.117 | 0.015 |
| MKN-45 | Gastric | 0.047 | 0.006 |
| PC3 | Prostate | 0.240 | 0.026 |
| DU145 | Prostate | 0.597 | 0.061 |

### Example 2

### Microsomal Stability in Human Liver Microsomes

Microsomal stability in vitro in different biologic media and species is ascertained when screening compounds to evaluate the rate at which a compound undergoes metabolism. Results in human liver microsomes for some of the compounds listed above are shown in the Table 3 below:

TABLE 3
Microsomal stability results for selected compounds

| Compound | % Metabolized (mean) |
|---|---|
| ACT-CK-050 | 99 |
| ACT-CK-142 | 99 |
| ACT-CK-143 | 34 |
| ACT-CK-146 | 85 |
| ACT-CK-147 | 91 |
| ACT-CK-148 | 54 |
| ACT-CK-151 | 91 |
| ACT-CK-152 | 47 |
| ACT-CK-153 | 99 |
| ACT-CK-154 | 99 |
| ACT-CK-155 | 69 |

### Example 3

### Cell Permeability

The determination of the permeability in Caco2 cells is widely used in screening compounds to assess intestinal drug transport and predict absorption rates. High permeability is determined as Pₐₚₚ>10×10⁻⁶ cm/s and suggests that a compound might be orally absorbed. Results for compound ACT-CK-050 below suggest that it has high permeability properties. Comparison of the apical to basal (A-B) and basal to apical (B-A) values also suggests that it is not actively effluxed out the cells and it is not a Pgp substrate.

TABLE 4
Permeability values in Caco2 cells

| | |
|---|---|
| | Pₐₚₚ |
| Compounds | (cm/sec 10⁻⁶) |

| | | |
|---|---|---|
| ACT-CK-050 | (A-B) | 46.1 |
| | (B-A) | 16.2 |

### Example 4

### Pharmacokinetics

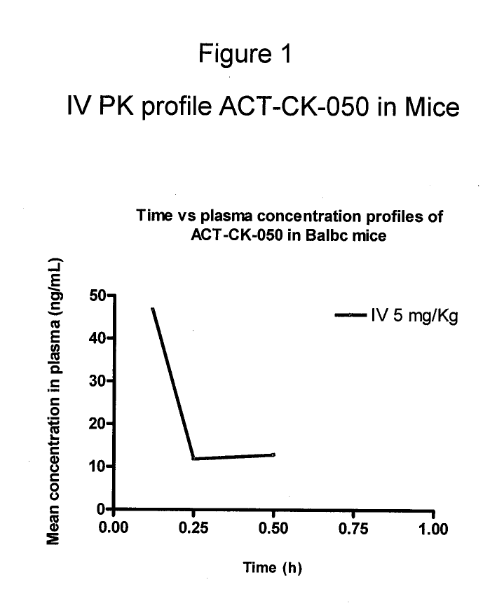

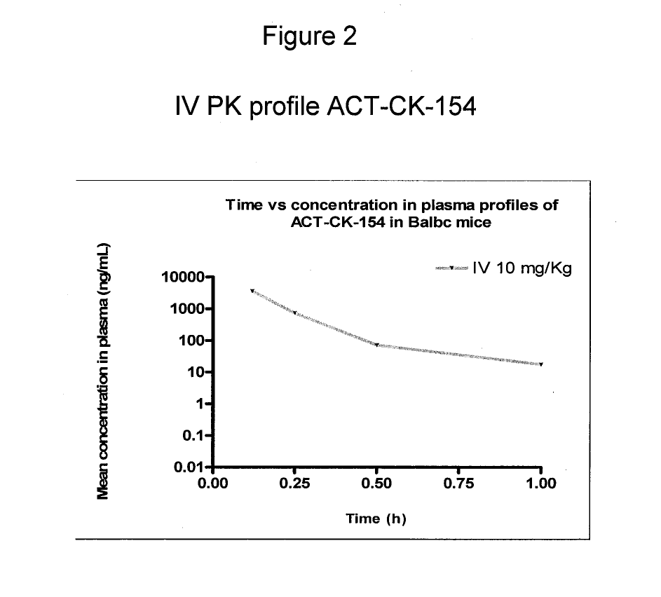

The pharmacokinetic parameters for several compounds listed above were determined in mice following IV (intravenous) or IP (intraperitoneal) administration. Briefly, six Balbc male mice 7 to 8 weeks old were used for each study. A dose of 5 mg/kg or 10 mg/kg was administered using a 5% DMSO/90% Captisol (20% in water) solution for ACT-CK-050 or FFF for ACT-CK-154. Blood samples were collected at different intervals. Plasma samples were extracted and analyzed using an LC-MS/MS method. Results are represented in FIGS. 1 and 2.

Plasma levels of ACT-CK-050 were too low and the compound was cleared too quickly to be able to determine the pharmacokinetic parameters under these experimental conditions. Parameters for ACT-CK-154 are shown in the Table 5 below:

TABLE 5
IV PK parameters for ACT-CK-154 in
Balbc male mice dosed at 5 mg/kg

| | T_{1/2b} | Cₘₐₓ | AUC_{0-inf} | CL | Vd |
|---|---|---|---|---|---|
| | (h) | (ng/mL) | (ng · h/ml) | (mL/min/kg) | (L/kg) |
| ACT-CK-154 | 0.15 | 3639 | 1293 | 129 | 1.66 |

### Example 5

### Efficacy Studies

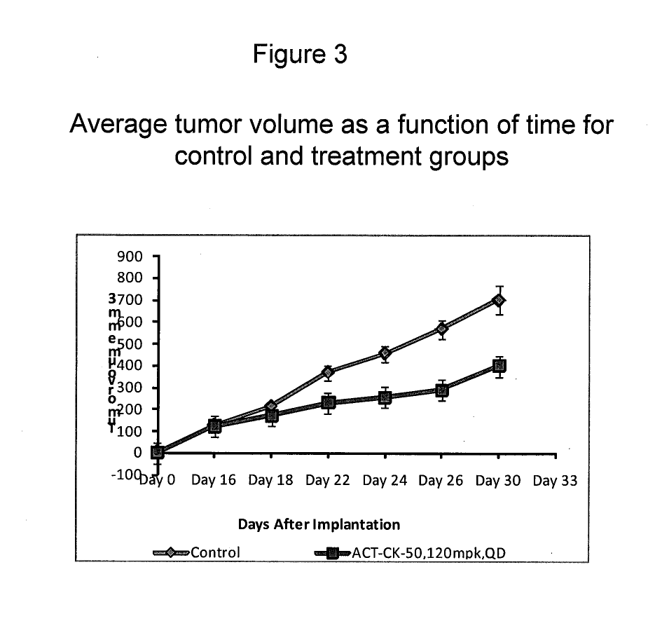

The activity of ACT-CK-050 was investigated in vivo in a Calu-6 xenograft tumor model. Briefly, Calu-6 lung tumor cells were grown in vitro using standard techniques. Three million cells were administered in a 1:1 mixture with Matrigel in the flank of female athymic nu/nu mice. Xenograft tumors formed and were monitored until an average size of 150 mm³. Mice were subsequently dosed IP with either the vehicle (control group, n=8) or ACT-CK-050 at 120 mg/kg daily (treatment group, n=8). Tumor volume was monitored in both groups and the average for both the control and treatment groups are shown in FIG. 3.

### Example 6

### Inhibition of Choline Kinase α

Choline Kinase α (CKα) was expressed and purified in order to determine if the compounds of the invention inhibits its enzymatic activity. CKα was prepared by expression in E. coli (Bl21(DE3)) and purified by GST column and column chromatography. SDS Page coumassie staining gels indicated that purity was high (>95%). The recombinant protein was pure and active as determined by the results of a kinase activity assay. The same assay was used to determine the inhibition of the protein and the results are shown in Table 6. These results confirmed that the compounds of the invention interact with CKα and modulate its activity.

TABLE 6
CKα Inhibition results for selected compounds

| Compounds | IC₅₀ (microM) |
|---|---|
| ACT-CK-050 | 20 |
| ACT-CK-109 | 18 |
| ACT-CK-157 | 50 |
| ACT-CK-153 | 30 |
| ACT-CK-170 | >100 |

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A compound or its tautomeric, enantiomeric or diastereomeric form or a pharmaceutically acceptable salt, prodrug, or metabolite thereof, said compound having the formula: 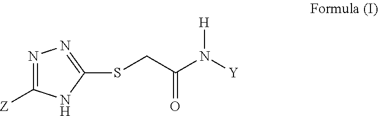
wherein:
Z is selected from the group consisting of: 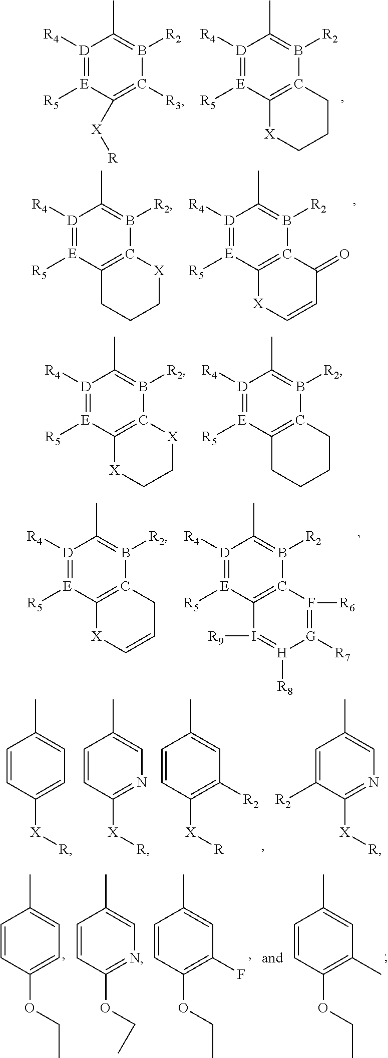
Y is selected from the group consisting of: 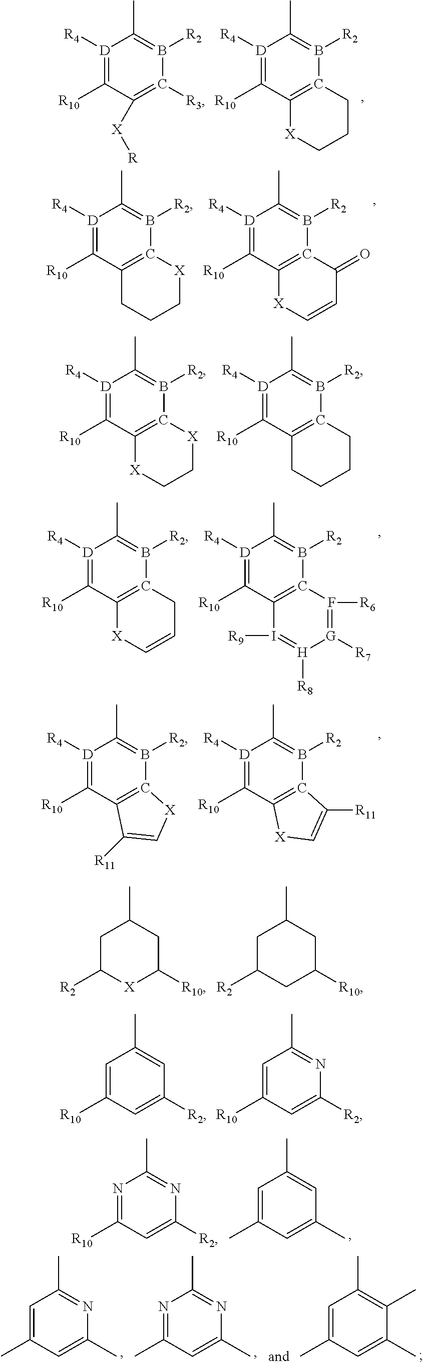
X is O, S, NH, or N-alkyl;
R is alkyl or substituted alkyl;
B, C, D, E, F, G, H, and I are independently selected from the group consisting of N or C substituted with one of R₂, R₃, R₄, R₅, R₆, R₇, R₈, or R₉, wherein if B, C, D, E, F, G, H, or I are N, then R₂, R₃, R₄, R₅, R₆, R₇, R₈, or R₉ represent the free electron pair at the N atom;
R₂, R₃, R₄, R₅, R₆, R₇, R₈, or R₉ are independently, when attached to an N atom, a free electron pair, or, when attached to C, selected from the group consisting of hydrogen, halogen, cyano, nitro, straight-chain or branched (C₁-C₆)-alkyl, straight-chain or branched (C₁-C₆)-alkyl substituted with one or more halogen atoms, straight-chain or branched (C₁-C₆)-alkoxy substituted with one or more halogen atoms, (C₂-C₆)-alkenyl, (C₂-C₆)-alkynyl, (C₃-C₈)-cycloalkyl, straight-chain or branched (C₁-C₆)-alkoxy, straight-chain or branched (C₁-C₆)-alkylenedioxy, (C₁-C₆)-alkoxycarbonyl, (C₁-C₆)-alkoxycarbonyloxy, (C₁-C₆)-alkylcarbonyl, (C₁-C₆)-alkylcarbonyloxy, (C₁-C₆)-alkylthio, (C₁-C₆)-alkylsulfinyl, (C₁-C₆)-alkylsulfonyl, carboxyl, (C₁-C₆)-alkyl carboxylate, carboxamide, N—(C₁-C₄)-alkyl-carboxamide, N,N-di-(C₁-C₄)-alkyl-carboxamide, (C₁-C₆)-alkoxy-(C₁-C₆)-alkyl, amino, mono-(C₁-C₆)-alkylamino, N,N-di-(C₁-C₆)-alkylamino, wherein two C₁-C₆-alkyl radicals together may form a ring, which optionally contains one or more of NH, N—(C₁-C₆)-alkyl, O or S, (C₆-C₁₄)-aryl, (C₆-C₁₄)-aryloxy, (C₆-C₁₄)-aryl-(C₁-C₄)-alkyl, (C₆-C₁₄)-aryl-(C₁-C₄)-alkoxy-(C₁-C₄)-alkyl, (C₁-C₆)-alkylcarbonyl, (C₁-C₆)-alkoxycarbonyl, hydroxyl, wherein two directly adjacent radicals may be attached to one another; and
R₁₀ is alkyl or substituted'alkyl.

2. The compound of claim 1, wherein Z is selected from the group consisting of:

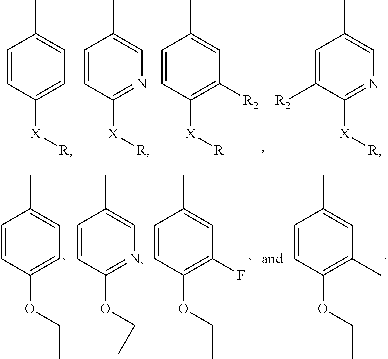

3. The compound of claim 2, wherein Z is selected from the group consisting of:

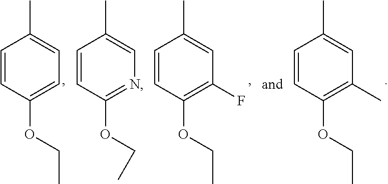

4. The compound of claim 1, wherein Y is selected from the group consisting of:

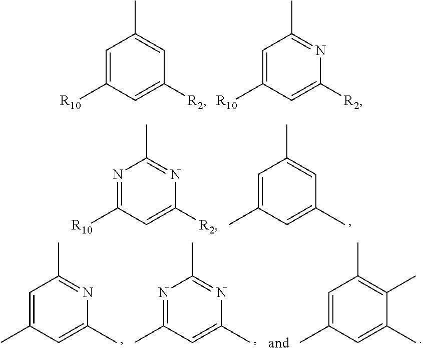

5. The compound of claim 4, wherein Y is selected from the group consisting of

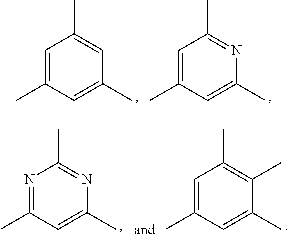

6. The compound of claim 1, wherein the straight-chain or branched (C₁-C₆)-alkyl substituted with one or more halogen atoms is trifluoromethyl.

7. The compound of claim 1, wherein the straight-chain or branched (C₁-C₆)-alkoxy substituted with one or more halogen atoms is trifluoromethoxy.

8. The compound of claim 1, wherein the straight-chain of branched (C₁-C₆)-alkoxy is methoxy.

9. The compound of claim 1, wherein the straight-chain or branched (C₁-C₆)-alkylenedioxy is methylenedioxy.

10. A pharmaceutical composition for the treatment of cancer comprising a compound according to claim 1 and at least one pharmaceutically acceptable carrier.

11. The pharmaceutical composition of claim 10, further comprising one or more additional chemotherapeutic agents.

12. A method of treating cancer comprising administering to a subject in need thereof an effective amount of a compound according to claim 1.

13. A method of inhibiting enzymatic activity of choline kinase in a cell, comprising administering an effective amount of a compound according to claim 1.

14. A method of treating a tumor, comprising administering to a subject in need thereof an effective amount of one or more compounds according to claim 1.

15. A compound selected from the group set forth in Table 1.

16. A compound selected from the group consisting of:
N-(3,5-Dimethyl-phenyl)-2-[5-(4-ethoxy-phenyl)-4H-[1,2,4]triazol-3-ylsulfanyl]-acetamide;
2-[5-(4-Methoxy-phenyl)-4H-[1,2,4]-triazol-3-ylsulfanyl]-N-(5-methyl-thiazol-2-yl)-acetamide;
N-(3,5-Dimethyl-phenyl)-2-[5-(1H-indol-2-yl)-4H-[1,2,4]-triazol-3-ylsulfanyl]-acetamide;
N-(3,5-Dimethyl-phenyl)-2-[5-(4-ethoxy-3-methyl-phenyl)-4H-[1,2,4]triazol-3-ylsulfanyl]-acetamide;
N-(3,5-Dimethyl-phenyl)-2-[5-(6-ethoxy-pyridin-3-yl)-4H-[1,2,4]triazol-3-ylsulfanyl]-acetamide;
N-(4,6-Dimethyl-pyridin-2-yl)-2-[5-(4-ethoxy-phenyl)-4H-[1,2,4]triazol-3-ylsulfanyl]-acetamide;
N-(4,6-Dimethyl-pyrimidin-2-yl)-2-[5-(4-ethoxy-phenyl)-4H-[1,2,4]triazol-3-ylsulfanyl]-acetamide;
N-(3,5-Dimethyl-phenyl)-2-[5-(4-ethoxy-3-fluoro-phenyl)-4H-[1,2,4]triazol-3-ylsulfanyl]-acetamide;
2-[5-(4-Ethoxy-3-fluoro-phenyl)-4H-[1,2,4]triazol-3-ylsulfanyl]-N-(2-hydroxy-3,5-dimethyl-phenyl)-acetamide; and
N-(3,5-Dimethyl-phenyl)-2-[5-(4-ethylsulfanyl-phenyl)-4H-[1,2,4]triazol-3-ylsulfanyl]-acetamide.
